(19) United States
(12) Patent Application Publication
(10) Pub. No.: US 2018/0258382 A1
(43) Pub. Date: Sep. 13, 2018

(54) APPARATUS FOR CULTURING AND INTERACTING WITH A THREE-DIMENSIONAL CELL CULTURE

(71) Applicant: UNIVERSITY OF FLORIDA RESEARCH FOUNDATION, INCORPORATED, Gainesville, FL (US)

(72) Inventors: BENJAMIN G. KESELOWSKY, GAINESVILLE, FL (US); THOMAS ETTOR ANGELINI, GAINESVILLE, FL (US); WALLACE GREGORY SAWYER, GAINESVILLE, FL (US); TAPOMOY BHATTACHARJEE, GAINESVILLE, FL (US)

(21) Appl. No.: 15/759,587

(22) PCT Filed: Sep. 16, 2016
(86) PCT No.: PCT/US2016/052102

§ 371 Date: Mar. 13, 2018

Related U.S. Application Data
(60) Provisional application No. 62/220,891, filed on Sep. 18, 2015.

(51) Int. Cl. C12M 1/12; G01N 33/50; C12M 3/00; C12M 1/26; C12N 5/00; C12M 1/00

(52) U.S. Cl. CPC C12M 25/16; G01N 33/5008; C12M 29/00; C12M 33/00; C12N 5/0062; C12M 21/08

(57) ABSTRACT

A biological cell and/or tissue growth apparatus operable to create, in a chamber of the apparatus, a three-dimensional (3D) cell culture and to interact with a 3D structure of the cells in the chamber to, for example, apply materials to and/or remove materials from the cells or the chamber. The apparatus may include equipment for printing the 3D cell culture in a 3D cell growth medium. The 3D cell growth medium may be a granular gel material that undergoes a temporary phase change in response to an applied stress, such as a thixotropic or “yield stress” material. The apparatus may be operated such that the 3D printing equipment “prints” the 3D cell culture by depositing cells at particular locations in the 3D cell growth medium.

### CROSS-REFERENCE TO RELATED APPLICATIONS

This application claims priority under 35 U.S.C. § 119(e) to U.S. Provisional Patent Application Ser. No. 62/220,891, titled “Apparatus for culturing and interacting with a three-dimensional cell culture” and filed Sep. 18, 2015, the entire contents of which are incorporated herein by reference.

### FIELD

Disclosed embodiments are related to an apparatus for culturing and interacting with cells and/or tissues of a three-dimensional cell culture, disposed in a three-dimensional growth medium that may be a thixotropic material.

### BACKGROUND

Conventional cell culture techniques involve growing cells on a two-dimensional (2D) substrate, such as a micro-well plate or a Petri dish. Such 2D cell cultures often include a growth medium disposed on the substrate to promote cell growth. However, the 2D environment of conventional cell cultures is often a poor substitute for the three-dimensional (3D) environment experienced by cells in vivo. For example, the behavior of a cell is often highly dependent on the microenvironment around the cell; in a 2D cell culture the microenvironment around the cell may be different than what a cell would experience in a 3D microenvironment.

Several techniques have been developed for 3D cell culture, including the use of hanging drop plates, magnetic levitation, or biomaterial scaffolds. However, these techniques are typically expensive and/or time consuming, and may be limited in the specific structures or geometries of tissues which may be grown and/or tested.

### SUMMARY

In on embodiment, there is provided an apparatus for culturing and interacting with biological cells and/or tissues. The apparatus comprises a chamber comprising a container holding a three-dimensional (3D) cell growth medium, the 3D cell growth medium being a thixotropic material, equipment to dispense biological cells and/or tissues at particular positions within the 3D cell growth medium in the container, and equipment to interact with the biological cells and/or tissues within the 3D cell growth medium in the container.

In another embodiment, there is provided a method of operating a bioreactor. The method comprises culturing cells and/or tissues in a 3D cell growth medium, the 3D cell growth medium being a thixotropic material, and, while the cells and/or tissues are disposed in the 3D cell growth medium, removing byproduct of cellular activity from the 3D cell growth medium.

In a further embodiment, there is provided a method of operating a bioreactor to expose cells to a material. The method comprises suspending cells at locations within a 3D cell growth medium contained in a container of the bioreactor, the 3D cell growth medium being a thixotropic material, operating the bioreactor to culture the cells suspended in the 3D cell growth material, operating the bioreactor to dispense a material into the 3D cell growth medium, and following dispensing of the material, evaluating the cells for an impact of the dispensed material on the cells.

It should be appreciated that the foregoing concepts, and additional concepts discussed below, may be arranged in any suitable combination, as the present disclosure is not limited in this respect. Further, other advantages and novel features of the present disclosure will become apparent from the following detailed description of various non-limiting embodiments when considered in conjunction with the accompanying figures.

### BRIEF DESCRIPTION OF DRAWINGS

The accompanying drawings are not intended to be drawn to scale. In the drawings, each identical or nearly identical component that is illustrated in various figures may be represented by a like numeral. For purposes of clarity, not every component may be labeled in every drawing. In the drawings:

FIG. 1 is a schematic representation of one embodiment of an apparatus for culturing and interacting with cells and/or tissues in a 3D cell growth medium.

FIG. 2 illustrates (a) one embodiment of an injector placing material along a complex path in a 3D cell growth medium, (b) the tip of an injector moving through hydrogel particles, and (c) the stress-strain response of a soft granular gel.

FIG. 3 is a schematic of a device for three-dimensionally printing according to some embodiments.

FIG. 4 is a flowchart of a process an apparatus may implement in some embodiments for culturing and interacting with cells in a 3D cell growth medium in a chamber of the apparatus.

FIG. 5 is a flowchart of one embodiment of a method for placing cells in a 3D cell growth medium.

FIGS. 6A-6D are schematic representations of embodiments of a 3D cell growth medium including a plurality of cell spheroids.

FIGS. 7A-7B illustrate examples of an apparatus for culturing and interacting with a 3D cell culture.

FIG. 8 is a flowchart of one embodiment of a method for preparing a 3D cell growth medium.

FIG. 9 illustrates hierarchical 3D vascular networks with variable aspect ratio that may be formed using techniques as described herein according to some embodiments.

FIG. 10 illustrates cell structures extruded into yield stress materials for exemplary experimental study according to some embodiments.

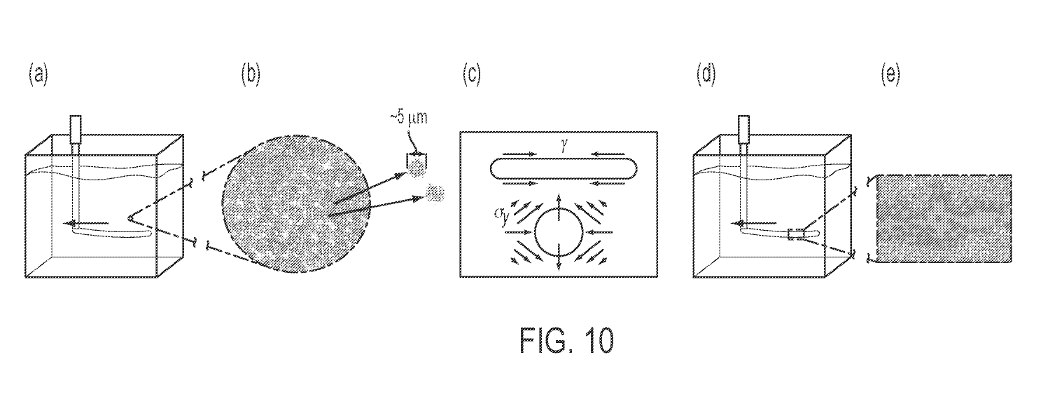

FIG. 11 illustrates a 3D extrusion system for exemplary experimental study according to some embodiments.

FIG. 12 is a diagram illustrating a system for three-dimensionally printing a tissue construct according to some embodiments.

FIG. 13 is a diagram illustrating an exemplary wound or tissue void of an organism and a tissue construct created to replace or repair the wound or tissue void according to some embodiments.

FIG. 14 is a diagram illustrating an exemplary tissue construct created from a three-dimensional model using commercially available software according to some embodiments.

FIG. 15 is a flowchart of a method for three-dimensionally printing a tissue construct according to some embodiments.

FIG. 16 is a diagram illustrating a computer system on which some embodiments may be implemented.

### DETAILED DESCRIPTION

Described herein are embodiments of a biological cell culturing and interaction apparatus. The apparatus operable to create, in a chamber of the apparatus, a three-dimensional (3D) cell culture arrange in a 3D cell growth medium. The apparatus is further operable to interact with a 3D culture of the cells in the medium and in the chamber to, for example, apply materials to and/or remove materials from the cells or the chamber.

In some embodiments, the apparatus may include equipment for depositing the cells at locations within a 3D cell growth medium, which holds the cells at the locations. The 3D cell growth medium may be a granular gel material that undergoes a temporary phase change in response to an applied stress, such as a thixotropic or “yield stress” material. The apparatus may be operated such that the 3D cell deposition equipment deposits cells at location within a container of the 3D cell growth medium, where the container is located in a chamber of the apparatus (which may be open or closed chamber). The cells may be cultured in the 3D cell growth medium to produce a 3D cell culture within the 3D cell growth medium.

In some embodiments, following deposition of the cells, the apparatus may be operable to interact with the cells in the chamber, within the 3D cell growth medium. For example, the apparatus may be operated to apply materials to and/or remove materials from the chamber. As another example, the apparatus may be operated to add or remove cells from the chamber. As another example, the apparatus may be operated to evaluate the cells within the chamber. As part of the interaction, the apparatus may exchange, add, or remove fluids within the chamber. In some embodiments the apparatus may be operable as a bioreactor.

In some embodiments, the apparatus may be operated to add/remove materials in different ways in different regions of the chamber, such as for different parts of a 3D cell culture or different 3D cell cultures disposed in the 3D growth medium and/or in the chamber. The different ways of interacting may include depositing different materials, or different concentrations of a material, in different regions of the chamber. For example, in some embodiments, the apparatus may be operated to create, in the 3D cell growth medium, different disparate segments or vessels in which different 3D cell cultures are grown (which may be the same cells, different cells, or any other suitable arrangement of cells). The apparatus may then be operated to apply different materials to the different segments or vessels.

The apparatus may be operable to add/remove different materials or combinations of materials. For example, the materials that the apparatus is operable to add/remove may include nutrients. In some embodiments the apparatus may be operated to apply nutrients to assist or induce growth of the cells. The nutrients may include 3D cell growth medium, or a nutrient material included in the 3D cell growth medium. The materials may also include pharmaceuticals, such as a pharmaceutical or combination of pharmaceuticals to be applied to the cells, which may include pharmaceuticals for which an impact on the cells is to be evaluated. The materials may also include byproducts of cellular activity of the cells, such as a case where the cells produce waste over time that is removed or where the cells produce a material (e.g., a pharmaceutical or other compound) that is harvested from the cells or from the chamber.

In addition to, or as an alternative to, applying or removing materials, the apparatus may be operated to add or remove the cells. For example, some or all of the cells, including samples from the cells or samples from different segments or vessels of the cells, may be removed from the chamber. The cells that are removed may be cells to be evaluated, such as by inspecting cells following application of a material (e.g., pharmaceutical).

In addition to or as an alternative to adding or removing materials or cells, the apparatus may be operated to evaluate the cells in the chamber and in the 3D cell growth medium. Evaluation of the cells may include inspection of one or more attributes of cells. Evaluation may also include assaying cells. For example, the apparatus may be operated to inspect attributes of individual cells or tissues, such as morphological attributes. As another example, the apparatus may be operated to inspect attributes of a population of cells or a tissue such as survival time or recovery time following dosing with a pharmaceutical or combination of pharmaceuticals. Any suitable assay or other evaluation of cells may be performed in embodiments, as embodiments are not limited in this respect.

The 3D cell growth medium with which the apparatus may be operated in some embodiments may provide various advantages in culturing cells as well as in applying or removing materials or cells or evaluating the cells. As described in detail below, the 3D cell growth medium may be a yield stress material that phase changes upon application of energy, such as upon application of a force. As discussed above, the apparatus may construct a 3D cell culture in the 3D cell growth medium and hold the cells in the 3D cell growth medium during culturing of the cells and interaction with the cells. Accordingly, the 3D cell growth medium in which the cells are disposed may perform the phase changes when a force is applied, which may have various benefits.

For example, the 3D cell growth medium may be advantageous for use with an apparatus, as described herein, in which cells are to be cultured over time, after which the apparatus may interact with the cells. As the cells grow over time, the volume of the cells in the 3D cell growth medium, including within a segment or vessel of the 3D cell growth medium, may grow over time. In traditional, inflexible support structures, the growth in volume of the cells may result in a compressive force on the cells if the cells expand to the size of a container in which they are held. In the case of the 3D cell culture created in a 3D cell growth medium, however, a yield stress of the material may be such that a force applied by the growing cells may be sufficient to trigger a phase change in the 3D cell growth medium, after which the 3D cell growth medium may distort to expand a volume in which the cells are disposed, to provide more space for the cells. The cells may therefore remain in the 3D cell culture in the 3D cell growth medium, and in the chamber of the apparatus, over time without substantial concern regarding a compressive force that may result from growth of the cells.

As another example, the 3D cell culture disposed within the 3D cell growth medium may be advantageous as it may allow interaction by the apparatus with the cells while the cells are disposed within the 3D cell growth medium and within the chamber of the apparatus. The apparatus may include equipment to interact with the cells, such as syringes to dispense or extract materials or cells. Prior to interaction by the apparatus with the cells, the interaction equipment may be disposed wholly apart from the 3D cell growth medium. To interact with the cells, the interaction equipment may contact and impose a force on the 3D cell growth medium. In an area that the interaction equipment imposes the force on the 3D cell growth medium, the 3D cell growth medium may change phase and permit the interaction equipment to penetrate the 3D cell growth medium, while other parts of the 3D cell growth medium, including adjacent parts of the 3D cell growth medium, remain do not change phase. Following the interaction, the interaction equipment may be extracted and the 3D cell growth medium may reshape to fill in the space previously-occupied by the interaction equipment. Accordingly, the 3D cell growth medium may permit dynamic interaction with the cells while they are disposed within the 3D cell growth medium. Alternatively, in some embodiments, the interaction equipment may be inserted into the chamber or into the 3D cell growth medium before, during, or after the 3D cell growth medium is created. For example, while the apparatus is being operated to deposit the cells in the 3D cell growth medium, or otherwise operated to create the 3D cell culture, the interaction equipment may be inserted into the 3D cell growth medium. The interaction equipment may also be dynamically removed from the 3D cell growth medium.

Embodiments may operate with any suitable interaction equipment, which may vary depending on the types of interactions to be performed, such as dispensing materials, removing materials, removing cells, evaluating cells, or other interactions. The interaction equipment may include syringes, pipettes, perfusion tubing, or other equipment. The interaction equipment may include equipment that does not directly contact the cells. For example, the tubing or syringes may not contact the cells. As another example, the chamber of the apparatus, or the container in the chamber in which the 3D cell growth medium is held, may include dispensers and outlets for adding or removing materials from the chamber/container, such as for adding or removing fluids. The equipment may include pumps or other equipment to inject or draw off fluids. The equipment may also include centrifuge equipment, to cause expulsion of fluids or other materials in the chamber. For example, the apparatus, the chamber of the apparatus, or the container in the chamber may, in some embodiments, be located within a centrifuge, which may be operated to spin the apparatus/chamber/container and cause materials therein to be expelled. In some embodiments that include pumps, centrifuges, or other equipment that may impose a force on the 3D cell growth medium, the equipment may be operated such that a force imposed may be below the yield stress of the 3D cell growth medium or below another force threshold. In addition, in some embodiments in which pumps, centrifuges, or other equipment is used to draw out material from the chamber or from the 3D cell growth medium, a filter-like membrane may separate the 3D cell growth medium and the 3D cell culture from an outflow of the container or chamber. The membrane may have a pore size or otherwise be arranged to prevent some content of the 3D cell growth medium from exiting the container in which the 3D cell growth medium is held. For example, where the 3D cell growth medium includes a hydrogel and a liquid cell growth material, the membrane may prevent the hydrogel from exiting the container.

While embodiments have been described in which an apparatus creates a 3D cell culture in a 3D cell growth medium, cultures the cells in the 3D cell growth medium, and interacts with the cells in the 3D cell growth medium all within a chamber of the apparatus, it should be appreciated that embodiments are not so limited. For example, an apparatus may support placement in a chamber of the apparatus of a 3D cell growth medium having cells already disposed therein, such as cells that were deposited in the 3D cell growth medium by another, potentially different apparatus. The apparatus may then interact with the cells within the 3D cell growth medium.

### 3D Cell Growth Medium and 3D Cell Culture Made in 3D Cell Growth Medium

The inventors have recognized and appreciated that a 3D cell growth medium may be provided using the materials and methods described herein. The inventors have recognized and appreciated that creating a 3D cell growth medium using the materials described herein may allow for cell growth environment which more closely mimics the complex in vivo growth environment compared to typical 2D cell culture techniques. For example, culturing cells in a 3D culture as described herein may facilitate cell-cell interactions and the induction of biological processes, including cellular differentiation. Nonetheless, those techniques may allow for easy placement and/or retrieval of groups of cells, which may enable rapid and/or high throughput testing. Such testing may reduce or eliminate the need for pre-clinical animal testing as part of new drug development. For example, may enable cancer cells to be grown in structures that mimic the dynamic environment in a cancerous tumor. Drugs may be applied to such tumors such that an indication of the efficacy of such drugs can be obtained in a fashion that is more reliable than using conventional in vitro test techniques.

Moreover, the inventors have recognized and appreciated that the 3D cell growth media described herein may allow for growing diverse cellular structures, including, but not limited to, spheroids, embryoid bodies, tumors, cysts, and microtissues, and may also be used to preserve the structure of cell-laden engineered tissue constructs.

In some embodiments, a 3D cell growth medium may comprise hydrogel particles dispersed in a liquid cell growth medium. The inventors have recognized and appreciated that any suitable liquid cell growth medium may be used; a particular liquid cell growth medium may be chosen depending on the types of cells which are to be placed within the 3D cell growth medium. Suitable cell growth medium may be human cell growth medium, murine cell growth medium, bovine cell growth medium or any other suitable cell growth medium. Depending on the particular embodiment, hydrogel particles and liquid cell growth medium may be combined in any suitable combination. For example, in some embodiments, a 3D cell growth medium comprises approximately 0.5% to 1% hydrogel particles by weight.

In accordance with some embodiments, the hydrogel particles may be made from a bio-compatible polymer.

The hydrogel particles may swell with the liquid growth medium to form a granular gel material. Depending on the particular embodiment, the swollen hydrogel particles may have a characteristic size at the micron or submicron scales. For example, in some embodiments, the swollen hydrogel particles may have a size between about 0.1 μm and 100 μm. Furthermore, a 3D cell growth medium may have any suitable combination of mechanical properties, and in some embodiments, the mechanical properties may be tuned via the relative concentration of hydrogel particles and liquid cell growth medium. For example, a higher concentration of hydrogel particles may result in a 3D cell growth medium 3D cell growth medium having a higher elastic modulus and/or a higher yield stress.

The inventors have recognized and appreciated that such tunability may be advantageous for controlling the environment around a group of cells placed in a 3D cell growth medium. For example, a 3D cell growth medium may have mechanical properties which are tuned to be similar to those found in vivo so that the cells 3D cell growth medium 3D cell growth medium may emulate the natural environment of the cells. However it should be understood that the mechanical properties of a 3D cell growth medium may not be similar to those found in vivo, or may be tuned to any suitable values, as the disclosure is not so limited.

According to some embodiments, a 3D cell growth medium may be made from materials such that the granular gel material undergoes a temporary phase change due to an applied stress (e.g. a thixotropic or “yield stress” material). Such materials may be solids or in some other phase in which they retain their shape under applied stresses at levels below their yield stress. At applied stresses exceeding the yield stress, these materials may become fluids or in some other more malleable phase in which they may alter their shape. When the applied stress is removed, yield stress materials may become solid again. Stress may be applied to such materials in any suitable way. For example, energy may be added to such materials to create a phase change. The energy may be in any suitable form, including mechanical, electrical, radiant, or photonic, etc.

The inventors have recognized and appreciated that providing a 3D cell growth medium made from a yield stress material, as described above, may enable facile placement and/or retrieval of a group cells at any desired location within the 3D cell growth medium 3D cell growth medium. For example, placement of cells may be achieved by causing a solid to liquid phase change at a desired location in a region of yield stress material such that the yield stress material will flow and be displaced when cells are injected or otherwise placed at the desired location. After injection, the yield stress material may solidify around the placed cells, and therefore trap the cells at the desired location.

However, it should be appreciated that any suitable techniques may be used to deposit cells or other biological materials within the 3D growth medium. For example, using a syringe, pipette or other suitable tool, cells may be injected into one or more locations in the 3D growth medium. In some embodiments, the injected cells may be shaped as a pellet, such as by centrifugation. However, it should be appreciated that a 3D growth medium as described herein enables injection of cells suspended in a liquid, which may avoid a centrifugation step in conducting tests.

Regardless of how cells are placed in the medium, the yield stress of the yield stress material may be large enough to prevent yielding due to gravitational and/or diffusional forces exerted by the cells such that the position of the cells within the 3D cell growth medium 3D cell growth medium may remain substantially constant over time. Since the cells are fixed in place, they may be retrieved from the same location at a later time for assaying or testing by causing a phase change in the yield stress material and removing the cells. As described in more detail below, placement and/or retrieval of groups of cells may be done manually or automatically.

A yield stress material as described herein may have any suitable mechanical properties. For example, in some embodiments, a yield stress material may have an elastic modulus between approximately 1 Pa and 1000 Pa when in a solid phase or other phase in which the material retains its shape under applied stresses at levels below the yield stress. In some embodiments, the yield stress required to transform a yield stress material to a fluid-like phase may be between approximately 1 Pa and 1000 Pa. When transformed to a fluid-like phase, a yield stress material may have a viscosity between approximately 1 Pa s and 10,000 Pa s. However, it should be understood that other values for the elastic modulus, yield stress, and/or viscosity of a yield stress material are also possible, as the present disclosure is not so limited.

In some embodiments, the yield stress may be tuned to match the compressive stress experienced by cell groups in vivo, as described above. Without wishing to be bound by any particular theory, a yield stress material which yields at a well-defined stress value may allow indefinite and/or unrestricted growth or expansion of a group of cells. Specifically, as the group of cells grows, it may exert a hydrostatic pressure on the surrounding yield stress material; this hydrostatic stress may be sufficient to cause yielding of the yield stress material, thereby permitting expansion of the group of cells. In such embodiments, the yielding of the yield stress material during growth of a group of cells may result in the yield stress material maintaining a constant pressure on the group of cells during growth. Moreover, because a yield stress material will yield when an applied stress exceeds the yield stress, a 3D cell growth medium 3D cell growth medium made from a yield stress material may not be able to apply a stress to a group of cells which exceeds the yield stress. The inventors have recognized and appreciated that such an upper bound on the stress applied to a group of cells may help to ensure that cells are not unnaturally constrained, damaged or otherwise altered due to the application of large compressive stresses.

According to some embodiments, a 3D cell growth medium 3D cell growth medium made from a yield stress material may yield to accommodate excretions such as fluids or other extracellular materials from a group of cells disposed within the 3D cell growth medium 3D cell growth medium. Without wishing to be bound by any particular theory, excretion of fluids or other materials from a group of cells may result in an increase in the pressure in the extracellular space; if the pressure exceeds the yield stress of the 3D cell growth medium 3D cell growth medium, the 3D cell growth medium 3D cell growth medium may yield to accommodate the excretions, and a group of cells may excrete fluids or other materials without restriction. Such an ability of a 3D cell growth medium 3D cell growth medium to accommodate cellular excretion may allow the 3D cell growth medium to more closely match an in vivo environment. Moreover, the inventors have recognized and appreciated that a 3D cell growth medium made from a yield stress material may allow for facile removal of cellular excretions for assaying, testing, or any other suitable purpose, as described in more detail below.

A group of cells may be placed in a 3D cell growth medium made from a yield stress material via any suitable method. For example, in some embodiments, cells may be injected or otherwise placed at a particular location within the 3D cell growth medium with a syringe, pipette, or other suitable placement or injection device. In some embodiments an array of automated cell dispensers may be used to inject multiple cell samples into a container of 3-D growth medium. Movement of the tip of a placement device through the 3D cell growth medium may impart a sufficient amount of energy into a region around the tip to cause yielding such that the placement tool may be easily moved to any location within the 3D cell growth medium. In some instances, a pressure applied by a placement tool to deposit a group of cells within the 3D cell growth medium may also be sufficient to cause yielding such that the 3D cell growth medium flows to accommodate the group of cells. Movement of a placement tool may be performed manually (e.g., “by hand”), or may performed by a machine or any other suitable mechanism.

In some embodiments, multiple independent groups of cells may be placed within a single volume of a 3D cell growth medium. For example, a volume of 3D cell growth medium may be large enough to accommodate at least 2, at least 5, at least 10, at least 20, at least 50, at least 100, at least 1000, or any other suitable number of independent groups of cells. Alternatively, a volume of 3D cell growth medium may only have one group of cells. Furthermore, it should be understood that a group of cells may comprise any suitable number of cells, and that the cells may of one or more different types.

Depending on the particular embodiment, groups of cells may be placed within a 3D cell growth medium according to any suitable shape, geometry, and/or pattern. For example, independent groups of cells may be deposited as spheroids, and the spheroids may be arranged on a 3D grid, or any other suitable 3D pattern. The independent spheroids may all comprise approximately the same number of cells and be approximately the same size, or alternatively different spheroids may have different numbers of cells and different sizes. In some embodiments, cells may be arranged in shapes such as embryoid or organoid bodies, tubes, cylinders, toroids, hierarchically branched vessel networks, high aspect ratio objects, thin closed shells, or other complex shapes which may correspond to geometries of tissues, vessels or other biological structures.

According to some embodiments, a 3D cell growth medium made from a yield stress material may enable 3D printing of cells to form a desired pattern in three dimensions. For example, a computer-controlled injector tip may trace out a spatial path within a 3D cell growth medium and inject cells at locations along the path to form a desired 3D pattern or shape. Movement of the injector tip through the 3D cell growth medium may impart sufficient mechanical energy to cause yielding in a region around the injector tip to allow the injector tip to easily move through the 3D cell growth medium, and also to accommodate injection of cells. After injection, the 3D cell growth medium may transform back into a solid-like phase to support the printed cells and maintain the printed geometry. However, it should be understood that 3D printing techniques are not required to use a 3D cell growth medium as described herein.

The inventors have recognized and appreciated that a 3D cell growth medium made from a yield stress material may also allow for facile retrieval of groups of cells from within the cell growth medium via a reversal of the steps used to deposit the cells. For example, cells may be removed by simply moving a tip of a removal device such as a syringe or pipette to a location where a group of cells is disposed, and applying suction to draw the cells from the cell growth medium. As described above, movement of the tip of the removal device through the 3D cell growth medium may impart sufficient energy to the material to cause yielding and accommodate removal of the cells from the 3D cell growth medium. Such an approach may be used, for example, as part of a test process in which multiple cell samples are deposited in 3D cell growth medium. Those deposited cells may be cultured under the same conditions, but different ones of the samples may be exposed to different drugs or other treatment conditions. One or more samples may be harvested at different times to test impact of the treatment conditions on the cells.

The inventors have recognized and appreciated that in some embodiments in which cells excrete fluids or other materials into an extracellular space, the excretions may be removed from the cell growth medium with similar methods while not removing the cells. For example, the 3D cell growth medium may support the cells and keep them substantially stationary when removing cellular excretions. In some embodiments, yielded 3D cell growth medium may flow to fill in space which was previously occupied by removed cells and/or cellular excretions.

In some embodiments, a 3D cell growth medium may be used to support and/or preserve the structure of a cell-laden engineered tissue construct. For example, a tissue construct including a scaffold or other suitable structure on which a plurality of cells is disposed may be placed into a 3D cell culture medium. The 3D cell culture medium may provide support to preserve a complex structure of the tissue construct while also providing a 3D environment for cell growth which may mimic that found in vivo.

According to some embodiments, a 3D cell growth medium may be prepared by dispersing hydrogel particles in a liquid cell growth medium. The hydrogel particles may be mixed with the liquid cell growth medium using a centrifugal mixer, a shaker, or any other suitable mixing device. During mixing, the hydrogel particles may swell with the liquid cell growth medium to form a material which is substantially solid when an applied shear stress is below a yield stress, as discussed above. After mixing, entrained air or gas bubbles introduced during the mixing process may be removed via centrifugation, agitation, or any other suitable method to remove bubbles from 3D cell growth medium.

In some embodiments, preparation of a 3D cell growth medium may also involve buffering to adjust the pH of a hydrogel particle and liquid cell growth medium mixture to a desired value. For example, some hydrogel particles may be made from polymers having a predominantly negative charge which may cause a cell growth medium to be overly acidic (have a pH which is below a desired value). The pH of the cell growth medium may be adjusted by adding a strong base to neutralize the acid and raise the pH to reach the desired value. Alternatively, a mixture may have a pH that is higher than a desired value; the pH of such a mixture may be lowered by adding a strong acid. According to some embodiments, the desired pH value may be in the range of about 7.0 to 7.4, or, in some embodiments 7.2 to 7.6, or any other suitable pH value which may, or may not, correspond to in vivo conditions. The pH value, for example may be approximately 7.4. In some embodiments, the pH may be adjusted once the dissolved CO₂ levels are adjusted to a desired value, such as approximately 5%

In one non-limiting example, a 3D cell growth medium comprises approximately 0.2% to about 0.7% by mass Carbopol particles (Lubrizol). The Carbopol particles are mixed with and swell with any suitable liquid cell growth medium, as described above, to form a 3D cell growth medium which comprises approximately 99.3% to about 99.8% by mass cell growth medium. After swelling, the particles have a characteristic size of about 1 μm to about 10 μm. The pH of the mixture is adjusted to a value of about 7.4 by adding a strong base, such as NaOH. The resulting 3D cell growth medium is a solid with a modulus of approximately 100-300 Pa, and a yield stress of approximately 20 Pa. When a stress is applied to this 3D cell growth medium which exceeds this yield stress, the cell growth medium transforms to a liquid-like phase with a viscosity of approximately 1 Pa s to about 1000 Pa s. As described above, the specific mechanical properties may be adjusted or tuned by varying the concentration of Carbopol. For example, 3D cell growth media with higher concentrations of Carbopol may be stiffer and/or have a larger yield stress.

### 3D Printing Equipment and Techniques for 3D Printing

As described above, the apparatus for culturing and interacting with cells within a 3D cell growth medium may include equipment for depositing cells and/or tissues at particular locations within the 3D cell growth medium, to construct a 3D cell culture in the 3D cell growth medium.

The inventors have recognized and appreciated that a high speed and high precision way to replace tissue may be provided using the 3D printing techniques described herein. The inventors have recognized and appreciated that creating a 3D tissue construct using the 3D printing techniques described herein may provide soft tissue, which may be used to replace missing or damaged tissue, with higher precision and scale and at higher speed than previously possible. Such a tissue construct may also be made on-demand and to a custom specification. Such a tissue construct may also be made of biocompatible materials and/or of cells such that the tissue construct may merge with a portion of an organism, such as a person.

Printing techniques as described herein may support “printing” with multiple types of biomaterials in the same construct, such that the tissue construct may merge with a portion of an organism with multiple types of tissue. For example, a deep wound might be repaired by a tissue construct that includes biomaterials compatible with multiple types of muscles. Further, printing techniques as described herein, by providing high precision may enable printing of small passageways that support the formation of vasculature and microvasculature when the construct merges with the organism.

The inventors have recognized and appreciated that by printing into a temporarily phase changed material (e.g., a thixotropic or “yield stress” material), a desired structure may be printed without having to print support material as well. Rather, the phase changed material may become the support material, by conforming to the printed volume and reverting to a phase that constrains the volume. The inventors have recognized and appreciated that this approach may decrease costs and manufacturing time as compared to conventional 3D printing systems, for which the surface tension between the printed material and the support material plays a key role in limiting the minimum feature size that can be printed. The printing may be achieved, for example, by injecting a second material into the phase changed material. The phase changed material may be temporarily created, for example, in a localized region of a yield stress material by energizing that region.

In some embodiments, the material injected into the temporarily phase changed material may be miscible with it. The inventors have recognized and appreciated that the minimum feature size that can be printed may be reduced by printing with such a miscible material. In cases where the printed material is immiscible with a supporting yield stress material, the competition between surface tension and yield stress may set a limit on printable feature size, comparable to that of traditional 3D printing where the Rayleigh-Plateau instability sets the minimum feature size. However, there may be no surface tension if the two materials are miscible, and the theoretical lower limit on printed feature size may be set by (a) the size of the microgel particles that constitute the yield stress material, (b) the size of the particles in the printed material, or (c) the size of the extrusion nozzle. Most “rapid prototyping” 3D printing systems use immiscible materials. The printed material is typically hydrophobic organic material, and the support material is typically water-soluble hydrophilic material. Thus, the minimum feature size of most commercially available 3D printers is limited by surface tension. The inventors have recognized and appreciated that printing particulate materials into particulate yield stress materials—both soluble in the same materials—may eliminate the surface tension limitation, which may side-step decades of technological challenges associated with surface wetting and interfacial energy. This improvement may be possible for “water-water” based printing, and “oil-oil” based printing; particulate aqueous suspensions can be printed into aqueous yield stress materials, and suspensions of oil-soluble particles can be printed into oil-based yield stress materials.

Yield stress materials may be solids or in some other phase in which they retain their shape under applied stresses at levels below their yield stress. At applied stresses exceeding the yield stress, these materials may become fluids or in some other more malleable phase in which they may alter their shape. When the applied stress is removed, yield stress materials may become solid again. Thus, yield stress materials may provide a self-healing, self-sealing support structure into which complex structures of arbitrary design can be printed. Many yield stress materials are particulate. For example, yield stress materials may include dense packs of microgels—microscopic particles made from a swollen crosslinked polymer network. Microgels can be made from aqueous, hydrophilic polymers or from hydrophobic polymers like PDMS. Other yield stress materials include clay and dense nanofiber suspensions.

Stress may be applied to such materials in any suitable way. For example, energy may be added to such materials to create a phase charge. The energy may be in any suitable form, including, mechanical, electrical, radiant or photonic, etc. The inventors have recognized and appreciated that substrates with complex geometry like tubes, toroids, spheres, cylinders, hierarchically branched vessel networks, high aspect ratio objects, and thin closed shells, may be challenging and time-consuming to fabricate with conventional methods, and that such substrates and structures with complex geometry may be printed more easily and more quickly using temporarily phase changed materials. Moreover, printing into a temporarily phase changed material may enable applications in which the printed structure does not solidify rapidly or at all, as it may not need to do so. In this way, printing stable structures made from nothing but water may be possible. The fluid may remain fluid forever because the temporarily phase changed material may hold the printed structure in place after printing or extrusion. By trapping the fluid or material within the temporarily phase changed materials, the effects of surface tension, gravity, and particle diffusion can be negated, which may enable the manufacturing of finely detailed delicate materials with nearly limitless aspect ratios. Moreover, the inventors have recognized and appreciated that structures can be “un-printed” using the same materials by reversing the path of the extrusion nozzle and reversing the flow direction.

The inventors have recognized and appreciated that a wide variety of materials may be used as temporarily phase changed materials, including silicones, hydrogels, colloidal particles, and living cells. Soft polymeric materials can be crosslinked into structures and removed from the temporarily phase changed material, while uncrosslinked particulate systems like colloids and cells can be left supported within the material for seemingly infinite times. The precision and level of detail achieved by writing within a temporarily phase changed material may be limited by the size of granules of the material, which may be made at micron and sub-micron sizes. This approach may aid in the development and manufacture of precise, hierarchical cell culture scaffolds, vascular networks, complex tissues, and, in some embodiments, entire organs.

The inventors have recognized and appreciated that, while most extant tissue printing techniques involve layer-by-layer deposition in a fluid bath with a solvent-casting method, in which the extruded material solidifies by the action of a compound in the bath (like alginate extruded into a calcium chloride bath), 3D printed tissues may be generated directly inside a “bath” of their nutrient medium with no intermediate solidification step or extracellular matrix using temporarily phase changed materials. This way, living tissue cells can be printed into arbitrary 3D structures, either with or without supplemental extracellular matrix material. The temporarily phase changed support material (e.g., energized yield stress material) may provide solidity, which may work without a “curing agent” to bolster the printed structure. Moreover, the inventors have recognized and appreciated that using temporarily phase changed materials may avoid the challenges of solvent-casting methods such as the nozzle frequently getting clogged as material solidifies before exiting into the bath. Cell growth medium may be used as the solvent for aqueous microgels, making, for example, a tissue culture yield stress matrix. As an alternative example, cells can be printed into an oil-based yield stress material, using interfacial tension to maintain a well-defined surface.

The 3D printing techniques may be applied in any of multiple ways. Specifically, the inventors have recognized and appreciated significant demands for a 3D substrate of controllable, well-defined topology and material property that will aid deconstructing the complexity of cell interactions with 3D culture systems. The inventors have also recognized and appreciated that printing into a temporarily phase changed material may enable engineering of an artificial 3D in vitro environment, which may satisfy the growing interest in isolating specific environmental cues (e.g., substrate curvature) that a 3D culture could provide.

The inventors have recognized and appreciated that by printing particulate suspensions—like cells or any commonly used inks in 2D and 3D printing—into a particulate, temporarily phase changed material such as a particulate yield stress material, the printed structure can be miscible with the support structure without loss of printing precision. This miscibility without loss of precision is possible because the printed structure may be instantly trapped in the surrounding yield stress material as soon as it is extruded. In the case of miscible components, since there may be no surface tension between printed material and support material, the fundamental limit of most 3D printing strategies may be sidestepped. There may be no driving force for the printed features to “ball up.” So, aqueous materials can be printed into aqueous supports, and oil-based materials can be printed into oil-based supports. These are in addition to any case of immiscible combinations, which may also be possible.

The inventors have recognized and appreciated that printing into a temporarily phase changed material may enable fabricating a 3D substrate or a cell encapsulating matrix of defined geometries. For example, yield stress materials may exhibit shear-thinning properties, characterized by viscosity reduction under stress and a return to their original solid-like state when stress is removed. This transient flow property may enable one to shape the material via simple shearing. According to some embodiments, the stress may be provided via an injector, such as a syringe needle, shearing across the yield stress material (referred to as the outer fluid) and the injection of an immiscible liquid (referred to as the inner fluid). The stress may yield a small region of the outer fluid, which may re-solidify when the motion of the needle halts and may trap a droplet of the inner liquid. Droplets of complex topology, e.g., toroidal or crescent-shaped droplets, can be generated by rotating the continuous phase around a central axis while extruding the inner liquid from an injection needle positioned slightly off-centered. The dimensions of the torus may be controlled by (1) varying the amount of liquid injected and (2) changing the position of the needle with respect to the center of rotation. Note when combined with horizontal movement of the needle, spiral-shaped droplets can also be made.

According to some embodiments, a 2D curved surface or surfaces may be fabricated with simultaneous cell seeding. Providing an oily yield stress fluid as the outer media and an aqueous dispersion of cells as the inner fluid, spherical or non-spherical droplets containing cells may be directly formed in a single-step process. Alternatively or additionally, 2D curved surfaces of tunable chemical and mechanical properties suitable for subsequent cell culture may be fabricated. In this case, the inner fluid may comprise common hydrogel or synthetic extracellular matrix materials (ECM) precursor solution. Solidification or gelation of the precursor solution may then be induced by ultraviolet (UV) or thermo-gelling processes, after which the solid may be isolated from the yield stress material and used as cell substrate.

According to some embodiments, a 3D cell encapsulation matrix of spherical or toroidal geometry may be fabricated. Cell entrapment technique may be used in conjunction with some embodiments simply by using a mixture of hydrogel precursor solution and cell dispersion as the inner fluid. Once polymerized via changes of physical or chemical conditions, depending on the materials of choice, the final structure may again be isolated from the outer yield stress material.

### Illustrative Examples of Cell Culturing and Interaction Apparatus

Turning now to the figures, specific non-limiting embodiments of an apparatus for 3D cell culturing and for interaction with the 3D cell culture are described in more detail.

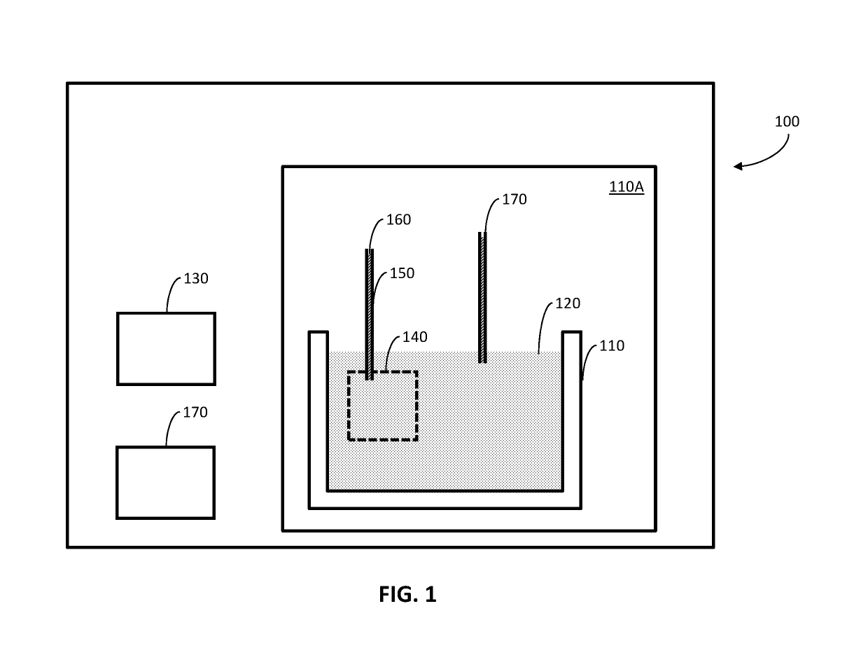

FIG. 1 depicts a schematic representation of one embodiment of an apparatus 100 for constructing a 3D cell culture in a 3D cell growth medium 120 and placing one or more groups of cells and/or tissues within the 3D cell culture created in the 3D cell growth medium 120. The apparatus 100 may include a container 110 located within a chamber 110A (which may be an open or closed chamber), a focused energy source 130, and an injector 150. The container 110 may hold the 3D cell growth medium 120. The focused energy source 130 may cause a phase change in a region 140 of the 3D cell growth medium 120 by applying focused energy to the region 140. The injector 150 may displace the 3D cell growth medium 120 with a material 160 which may include a plurality of cells.

According to some embodiments, the container 110 may be a tub, a bowl, a box, or any other suitable container for the 3D cell growth medium 120. As described above, the 3D cell growth medium 120 may include a thixotropic or yield stress material, or any material suitable for temporary phase changing. In some examples, the thixotropic or yield stress material may include a soft granular gel. The soft granular gel may be made from polymeric hydrogel particles swelled with a liquid cell culture medium. Depending on the particular embodiment, the hydrogel particles may be between 0.5 μm and 50 μm in diameter, between about 1 μm and 10 μm in diameter, or about 5 μm in diameter when swelled.

As discussed briefly above, in addition to equipment for creating a 3D cell culture in the 3D cell growth medium by dispensing cells in the 3D cell culture, the apparatus 100 may include equipment 170 for interacting with the cells.

The interaction equipment 170 may include equipment for dispensing one or more materials in the chamber 110A or in the 3D cell growth medium 120, which may include dispensing the material(s) to contact the cells or dispensing the material in the chamber 110A or in the 3D cell growth medium 120 after which the material may diffuse in the 3D cell growth medium. In cases in which material is dispensed, different materials or different combinations of materials may be dispensed at different locations in the 3D cell growth medium, which may include dispensing different materials to different cells or tissues, such as different cells that are segmented from one another in the 3D cell growth medium. Different materials may be dispensed to different cells as part of, for example, evaluating impacts of different materials on the cells. The materials that are dispensed may include nutrients, pharmaceuticals, detergents, fixatives, stains, or other materials.

The interaction equipment 170 may additionally or alternatively include equipment to remove materials from the chamber 110A and/or the 3D cell growth medium 120. For example, the equipment 170 may include equipment to remove waste materials produced by the cells, remove previously-added materials, remove byproduct materials created by the cells that are to be harvested, or remove other materials from the chamber 110A and/or 3D cell growth medium 120.

The interaction equipment 170 may additionally or alternatively include equipment to remove cells from the chamber 110A and/or the 3D cell growth medium 120. For example, cells that have been grown in the 3D cell growth medium 120 may be harvested from the medium 120. The cells may be harvested for evaluation.

The interaction equipment 170 may additionally or alternatively include equipment to evaluate cells within the chamber 110A and/or 3D cell growth medium 120. For example, the equipment may include equipment to inspect the cells and attributes of the cells. For example, morphological attributes of the cells or of the tissues may be evaluated, or attributes of a population of cells or of the tissues such as a survival time or a recovery time following exposure to a material (e.g., a dispensed material) may be evaluated. The evaluation equipment may include imaging equipment, including equipment to perform imaging following dispensing of a material (e.g., a stain and/or fixative).

The interaction equipment 170 may include equipment (including examples of equipment discussed above) that is used to assay the cells. The interaction equipment 170 may assay the cells within the chamber 110A and, in some cases, within the 3D cell growth medium 120.

In embodiments in which the interaction equipment 170 includes equipment to interact with specific cells within the chamber 110A, or interact with specific cells or parts of the chamber 110A differently, the interaction equipment 170 may include equipment to position (e.g., translate in two or three dimensions and/or rotate) components of the interaction equipment 170 within the chamber 170.

The interaction equipment 170 may include syringes, pipettes, perfusion tubing, pumps (including peristaltic pumps), centrifuges, or other equipment to perform the types of interactions described above, or other interactions that may be performed within a bioreactor. The interaction equipment 170 may include fluid exchange equipment to add, remove, or exchange fluids in the chamber 110A or the 3D cell growth medium 120 of the apparatus 100. The interaction equipment may further include imaging equipment or other equipment for performing inspections of cells within the 3D cell growth medium.

In embodiments in which one or more pumps are used to draw out material from the 3D cell growth medium, the pump(s) may be installed on one or more sides of a container holding the 3D cell growth medium in the chamber, or on one or more sides of the chamber. For example, the pump(s) may be arranged on a bottom of the container/chamber. The pumps—which may be peristaltic pumps or vacuum pumps, or other pumps—may draw material out of the 3D cell growth medium on one side. Other interaction equipment to dispense material may be arranged on another side of the container/chamber, such as an opposite side (e.g., top, where the outflow for the pump(s) is on a bottom), and may dispense material such as nutrients, pharmaceuticals, or other materials at the other side.

In embodiments in which one or more centrifuges are used to draw material from the 3D cell growth medium, the apparatus or a part of the apparatus, such as the chamber or the container holding the 3D cell growth medium, may be disposed within the centrifuge. The centrifuge may therefore impose a force on the entirety of the 3D cell growth medium, with the 3D cell culture disposed therein. The container for the 3D cell growth medium, or chamber in which the container is disposed, may include an outflow for materials that are expelled through action of the centrifuge. The outflow may be located on a side of the container/chamber in which centrifugal force with expel the material from the outflow. Other interaction equipment to dispense material may be arranged on another side of the container/chamber, such as an opposite side (e.g., top, where the outflow is on a bottom), and may dispense material such as nutrients, pharmaceuticals, or other materials at the other side.

In some embodiments that include an outflow from the container or chamber, the outflow or an opening of the outflow may include a filter-like membrane. The membrane may enable some materials to pass but block others. For example, in embodiments in which a 3D cell growth medium includes a hydrogel and a cell growth medium, the membrane may have a pore size or otherwise be arranged to prevent the hydrogel from passing through the membrane, and therefore keep the hydrogel in the container/chamber despite the pump or centrifuge, or other equipment, operating to draw material through the outflow.

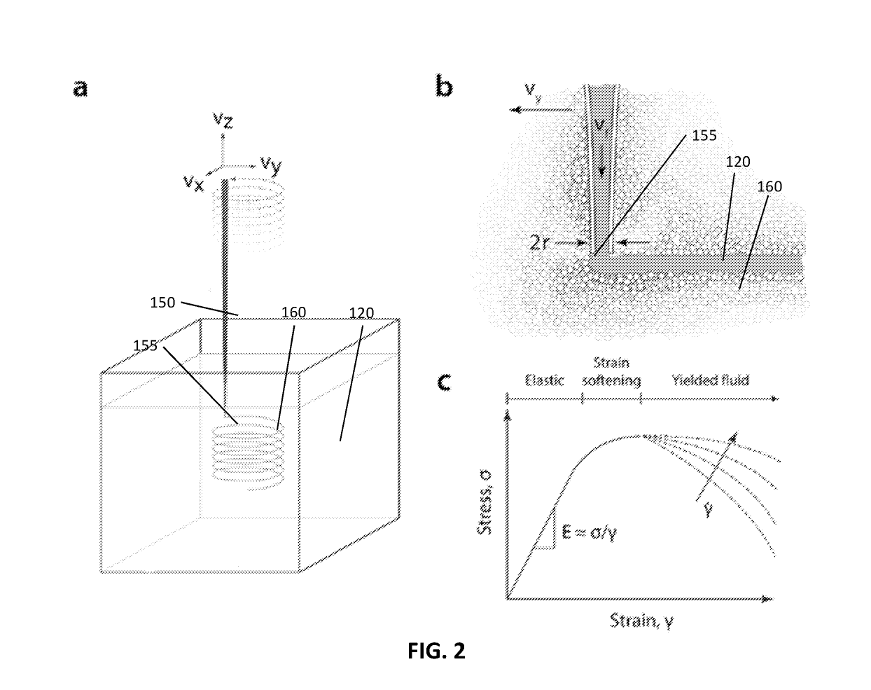

FIG. 2 provides more detail regarding some implementations of an apparatus 100 incorporating 3printing equipment to dispense cells at locations within a 3D cell growth medium, to form a 3D cell culture. FIG. 2 illustrates (a) an injector 150 comprising a capillary with a microscale tip 155 sweeping out a complex pattern in space as a material 160 is injected into a 3D cell growth medium 120. Arbitrary aspect ratio patterns can be generated because the structure itself may not need to solidify or generate any support on its own. Additionally, FIG. 2 illustrates (b) the tip 155 traversing solidly packed hydrogel particles which comprise the 3D cell growth medium 120; movement of the tip 155 may cause the particles to fluidize and then rapidly solidify, leaving a drawn cylinder in its wake. FIG. 2 also illustrates (c) the soft granular gel medium exemplarily as a yield stress material, which may elastically deform at low shear strains, soften at intermediate strains, and fluidize at high strains.

According to some embodiments, the focused energy may include mechanical energy, such as kinetic energy due to displacement of the injector 150 relative to the first material 120. In this example, the focused energy source 130 may include the injector 150. According to some embodiments, the injector 150 may include a fine hollow tip, which may carefully trace out spatial paths within the 3D cell growth medium 120 while injecting the material 160. The movement of the tip may locally yield and fluidize the 3D cell growth medium 120 at the point of injection (i.e., in the region 140). Another example of mechanical energy may include ultrasonic pressure waves. Alternatively or additionally, the focused energy may include radiant energy, such as radio frequency radiation, which may be directed into the region 140. It should be appreciated that movement of the injector may be performed manually (e.g. “by hand”) or may be automated (e.g. computer or machine controlled). Additionally or alternatively, the focused energy source 130 may cause a phase change in the region 140 of the 3D cell growth medium 120 to allow removal of material 160, including cells and or cellular excretions, as discussed above.

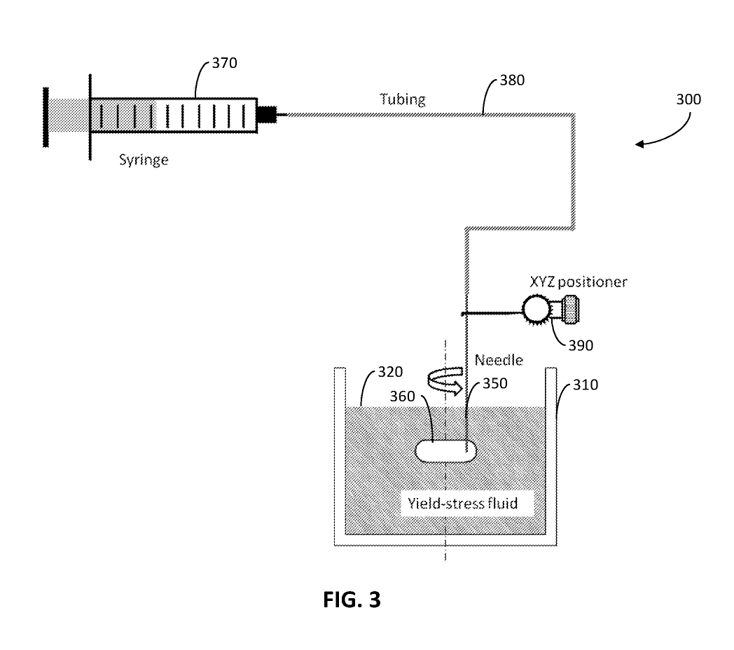

FIG. 3 illustrates further details of some embodiments of an apparatus 100 for culturing and/or interacting with cells in a 3D cell growth medium. The example of FIG. 3 includes additional details regarding 3D printing equipment that may be included in an apparatus to print a 3D cell culture in the 3D cell growth medium. FIG. 3 illustrates an apparatus 300 for three-dimensional printing. The apparatus 300 may include a housing or container 310, a needle 350, a syringe 370, and tubing 380. The housing 310 may hold a first material 320, which may be a 3D cell growth medium. The needle 350 may displace the first material 320 with a second material 360, which may include cells. The tubing 380 may be connected to an output of the syringe 370 and an input of the needle 350. The syringe 370 may include an amount of the second material 360, which it may inject via the tubing 380 and the needle 350 into the first material 320.

According to some embodiments, the apparatus 300 may include a platform (not shown) that may cause relative displacement between the first material 320 and the needle 350. Additionally, the relative displacement between the first material 320 and the needle 350 may comprise relative rotation between the first material 320 and the needle 350, as shown in FIG. 3. This relative rotation between the first material 320 and the needle 350 may comprise rotation about an axis of the first material 320, also shown in FIG. 3. According to some embodiments, the platform may cause the relative displacement between the cartridge 310 and the needle 350 at a displacement rate faster than a characteristic breakup time of a jet of the second material 360.

According to some embodiments, the apparatus 300 may further include a positioner or actuator 390. The positioner 390 may cause relative displacement between the needle 350 and the first material 320. For example, the positioner 390 may position the needle 350 three-dimensionally so that the second material 360 enters the first material 320 at the desired locations. The positioner 390 may also be used in conjunction with the platform to create specific shapes as the platform and positioner 390 each cause displacement simultaneously. For example, the platform may cause relative rotation between the first material 320 and the needle 350 while the positioner 390 may displace the needle 350 up and down, side to side, back and forth, and so on, creating any shape desired. Alternatively or additionally, the motion of the needle 350 may be synchronized with the motion of the positioner 390.

According to some embodiments, during fluid infusion, a liquid jet may be stretched by the continuous rotating motion of the outer fluid, similar to that of liquid co-flow. The principle of forming an enclosed curved jet (or toroidal droplet) inside a yield stress outer fluid may be similar to that of forming such droplets in simple Newtonian liquids (E. Priam et al. (2009), Generation and stability of toroidal droplets in a viscous liquid, Phys. Rev. Lett. 102, 234501): to perform a full rotation faster than the characteristic breakup time of the liquid jet. The temporarily phase changed portion or region of the first material 220, which may effectively be “solidified” under static conditions, may allow further stabilizing of the non-spherical geometry. For example, yield stress material may be immiscible with the inner fluid, preferably biocompatible, and may provide optical clarity as well as tunable mechanical properties.

Illustrative Methods for Operating Apparatus For Culturing and Interacting with Cells in a 3D Cell Growth Medium

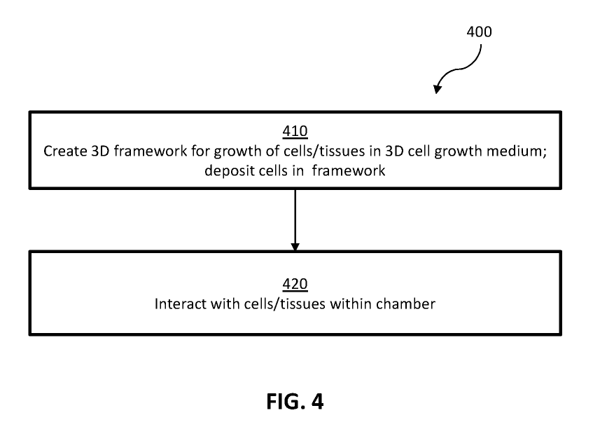

It should be appreciated from the foregoing that the apparatus described herein may be operated to three-dimensionally print or otherwise position cells in a desired position or pattern within a matrix created in and from a 3D cell growth medium as described herein. In addition, the apparatus may be operated to then interact with the cells. The apparatus may print and then interact with the cells in the 3D cell growth medium within a chamber (open or closed) of the apparatus. A method for operating the apparatus is illustrated in FIG. 4.

The process 400 begins in block 410, in which the apparatus creates, in a 3D cell growth medium, a 3D cell culture of cells by depositing the cells in the 3D cell growth medium in a desired position, pattern, or shape.

In block 420, the apparatus interacts with the cells within a chamber of the apparatus, such as within the 3D cell growth medium. To interact with the cells, the apparatus may dispense or remove materials, remove cells, evaluate tissues, or perform other types of interactions as described above.

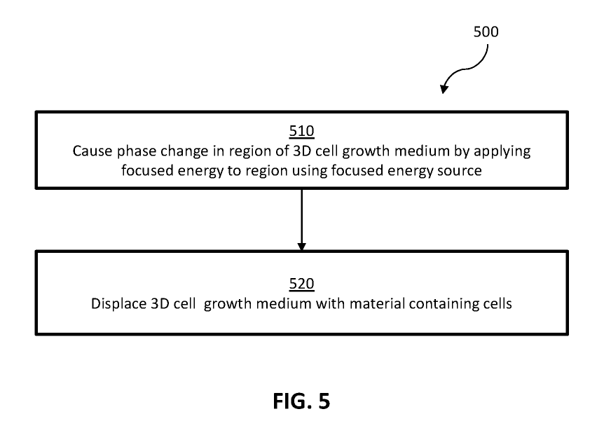

An example of a process by which the apparatus may be operated to print or otherwise position cells in the 3D cell growth medium is illustrated in FIG. 5. The method 500 begins at act 510, at which a phase change may be caused in a region of a 3D cell growth medium by applying focused energy to the region using a focused energy source. The 3D cell growth medium may be a material which may undergo a change from a less fluid to a more fluid state upon introduction of energy. In act 520, cells may be placed in the 3D cell growth medium by displacing the 3D cell growth medium with a material containing cells.

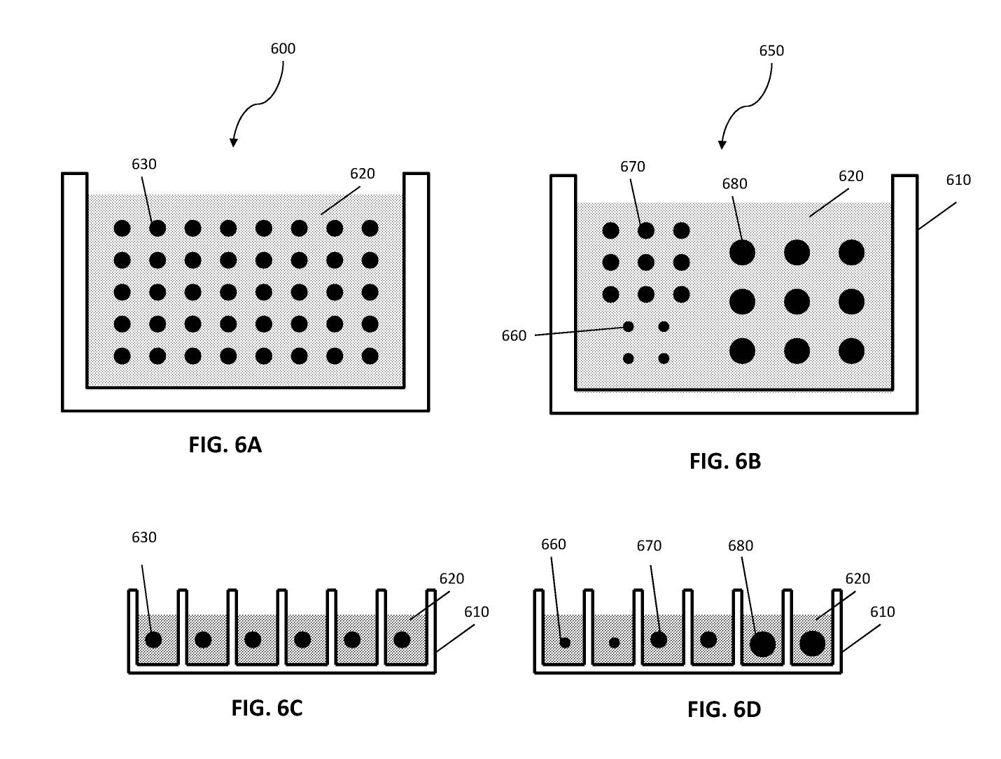

FIG. 6A depicts a cross sectional view of one embodiment of a 3D cell culture 600 including a 3D cell growth medium 620 disposed in a container 610. A plurality of spheroids 630 comprising one or more cells is arranged in the 3D cell growth medium 620. In the depicted embodiment, the spheroids 630 are approximately the same size and are spaced evenly spaced apart. In some embodiments, the spheroids may not all have the same size and/or spacing. For example, the FIG. 6B depicts another embodiment of a 3D cell culture 650 including small spheroids 660, intermediately sized spheroids 670, and large spheroids 680. In view of the above, it should be understood that cells spheroids of cells may have any suitable combination of sizes and/or spacing. Although spheroids are depicted, it should be understood that groups of cells may not be spheroid, and may be embryoid, organoid, or have any other suitable shape, as the disclosure is not so limited.

FIGS. 6A and 6B Figures illustrate the generation of multiple cell clusters, here shown as spheres, in the same vessel. FIGS. 6C and 6D illustrate the generation of multiple identical spheres or spheres of various sizes in numerous individual vessels. Vessels as illustrated may be formed in a tray 610 or other suitable carrier to facilitate high throughput testing. However, it should be appreciated, that any suitable vessel or vessels may be used.

Regardless of the type of vessel used, once the cells are deposited, the medium containing the cells may be incubated in diverse environments which may alter its chemical properties and in turn modify the growth environment of the 3D cultures contained within. For example, cells in the medium may be incubated in low oxygen or hypoxic environments.

It should be appreciated that one or more compounds may be deposited in conjunction with and/or adjacent to cells. For example, soluble, non-cellular components could be deposited in conjunction with the cells. These might include structural proteins (e.g. collagens, laminins), signaling molecules (growth factors, cytokines, chemokines, peptides), chemical compounds (pharmacologic agents), nucleic acids (e.g. DNA, RNAs), and others (nano-particles, viruses, vectors for gene transfer).

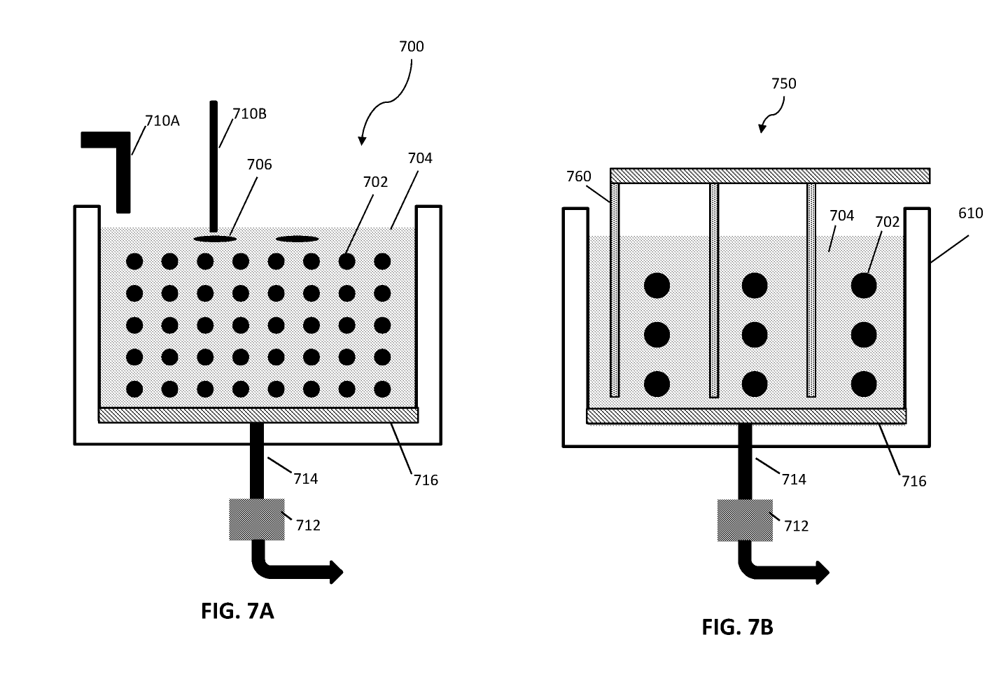

FIGS. 7A-7B illustrate examples of a cell culture and interaction apparatus, including examples of interaction equipment of such an apparatus.

FIG. 7A illustrates an apparatus 700 in which biological cells 702 are suspended at specific locations within a 3D cell growth medium 704. The apparatus includes interaction equipment 710A and 710B to dispense material into the 3D cell growth medium 704. Equipment 710A may dispense a cell growth material that, when combined with a hydrogel, forms the 3D cell growth material 704. The equipment 710A may dispense the cell growth material to supply nutrients as cells 702 absorb and use the cell growth material from the 3D cell growth material 704. Equipment 710B may also dispense material, such as by dispensing drug-loaded controlled release materials 706 into the 3D cell growth material 704. The controlled release materials 706 may diffuse through the 3D cell growth medium 704 to be absorbed by the cells 702.

Apparatus 700 may further include interaction equipment to remove fluids from the 3D cell growth material 704. As illustrated in FIG. 7A, the apparatus 700 may include a pump (e.g., a vacuum pump) 712, which may draw fluids out of the 3D cell growth material 704 via an outflow 714. In some embodiments, as illustrated in FIG. 7A, the apparatus 700 may include a filter-like membrane 716, which may permit some materials to pass into the outflow 714 but may block a hydrogel of the 3D cell growth material 704 or other materials from passing.

FIG. 7B illustrates another example of an apparatus 750, including different interaction equipment. Equipment and materials of the example of FIG. 7B that are the same as equipment/materials of FIG. 7A share the same reference numbers. The example of FIG. 7B illustrates perfusion tubing 760 to permit dispensing of one or more materials into the 3D cell growth material 704. Three perfusion tubes are illustrated. The same materials may be dispensed from each tube 760, or different materials may be dispensed. The materials that may be dispensed include a cell growth material, pharmaceuticals, or other compounds.

The equipment 710B and 760 of the examples of FIGS. 7A and 7B may be operated, in some embodiments, to dispense materials at particular locations within the 3D cell growth medium 704 and, in some embodiments, may be operated to dispense materials to form a concentration gradient of the materials across the 3D cell growth medium 704. By forming a gradient, different cells 702 may be exposed to different concentrations of a material. Following exposure, the cells 702 may be inspected (within or outside of the 3D cell growth medium 704) to determine an impact of different concentrations of the materials on the cells 702.

In some embodiments, as discussed above, the equipment 710B and 760 of FIGS. 7A and 7B may be dynamically inserted and removed from the 3D cell growth medium 704, while the cells 702 are cultured in the 3D cell growth medium 704.

In the examples of FIGS. 7A and 7B, the pump 712 may be used to remove materials from the 3D cell growth medium 704 for any suitable purpose. For example, the pump 712 may be operated to remove a byproduct of cellular activity, including waste generated by the cells or a protein or other byproduct of cellular activity that is to be harvested. As another example, the pump 712 may impose a force on the 3D cell growth medium 704 so as to draw materials (e.g., materials dispensed by equipment 710A, 710B, 760) through the 3D cell growth medium 704. While a pump 712 is shown applying such a force in the examples of FIGS. 7A and 7B, in other embodiments the source of the force may be a centrifuge spinning the apparatus 700, 750, or gravity, or any other suitable source of a force.

### Method of Preparing 3D Cell Growth Medium

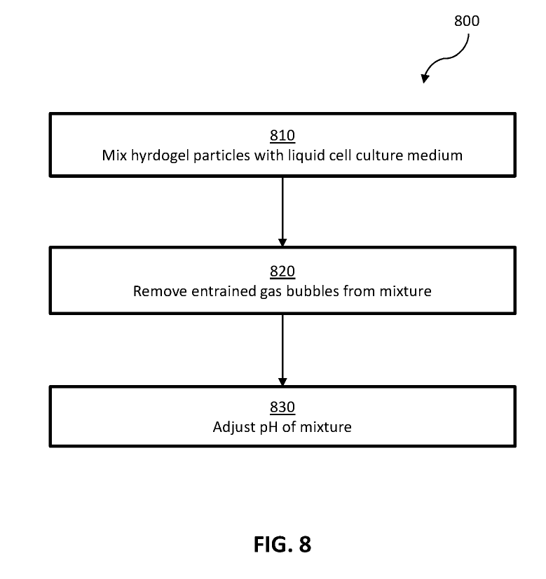

A method 800 for preparing a 3D cell growth medium is illustrated in FIG. 8. The method may be performed by a 3D cell growth and interaction apparatus as described herein, to create the 3D cell growth medium for use as described above. Alternatively, the method may be performed separate from the apparatus (by another apparatus or by a human), after which the 3D cell growth medium may be supplied to the apparatus.

The method 800 begins at act 810, at which hydrogel particles are mixed with a liquid cell culture medium. Mixing may be performed with a mechanical mixer, such as a centrifugal mixer, a shaker, or any other suitable mixing device to aid in dispersing the hydrogel particles in the liquid cell culture medium. During mixing, the hydrogel particles may swell with the liquid cell culture medium to form a granular gel, as discussed above. In some instances, the mixing act 810 may result in the introduction of air bubbles or other gas bubbles which may become entrained in the gel. Such entrained gas bubbles are removed at act 820 via centrifugation, gentle agitation, or any other suitable technique. The pH of the mixture may be adjusted at step 830; a base may be added to raise the pH, or alternatively an acid may be added to lower the pH, such until the pH of the mixture reaches a desired value. In some embodiments, the final pH value after adjustment is about 7.4.

It should be understood that the embodiments of 3D cell growth media described herein are not limited to any particular types of cells. For example, various embodiments of 3D cell growth media may be used with animal, bacterial, plant, insect, or any other suitable types of cells.

### Exemplary Operation Using 3D Printing Techniques

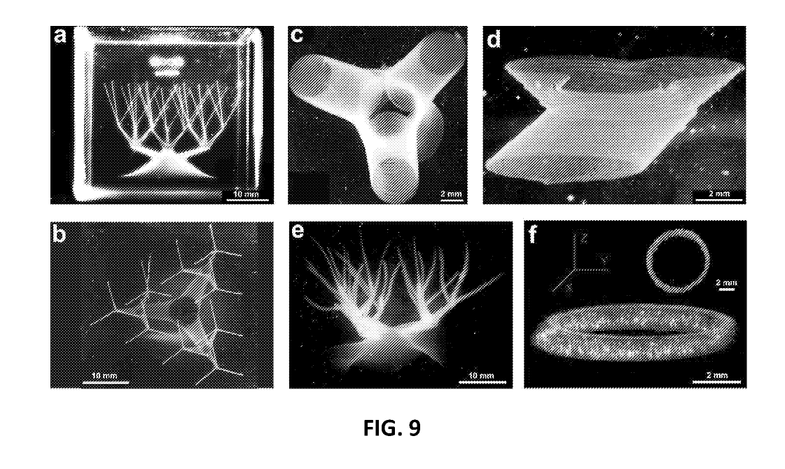

FIG. 9 illustrates an example of a structure that 3D printing techniques for dispensing cells in a 3D cell culture, as described herein, might be used create in some scenarios. As should be appreciated from the foregoing, equipment for dispensing cells into a 3D cell growth medium may be operable to dispense one or more types of cells in various arrangements and, including as a vascular network and as one or more tissues.

FIG. 9 illustrates exemplary hierarchical 3D vascular networks with variable aspect ratio. (a) A continuous network of hollow vessels is generated in which a large vessel branches to three smaller vessels, each branching to even smaller vessels, and so on, resulting in a single vascular network with features spanning about three orders of magnitude in size and many orders of magnitude in aspect ratio. A mixture of photocrosslinkable PVA and fluorescent microspheres is used for writing the structure. (b) The same network in (a) is shown from the top. (c) A high resolution photo of truncated vessels around a single junction show that the features are hollow with extremely thin walls. Single traces of the printed material can be seen, which have a diameter of approximately 100 micrometers. (d) The same junction from (c) is shown from the side without the top three vessels, demonstrating that concave and convex curvatures can be created in single stable structures. (e) The 3D vascular network is crosslinked, removed from the granular gel medium, and photographed while freely floating in water. (f) This entire vascular network was also created from human aortic endothelial cells (HAECs), written into granular gel medium permeated with cell growth media. However, the dielectric constant of the resulting structure is so close to the granular gel medium, and the features are so fine, the resulting structure cannot be seen in photographs. However, with fluorescently labeled cells, a portion of the structure may be measured using confocal fluorescence microscopy. The tilted tubular structure that forms the base in (d) can be seen, here made from fluorescently labelled HAECs. The image is a maximum intensity projection along a skewed direction, and the inset is the XY slice corresponding to the top of the tubular structure. The inventors have monitored cells in printed structures over the course of several weeks, finding no signs of toxicity.

### Exemplary Experimental Study of 3D Printing Techniques Using Biological Cells

Described below are specific examples of techniques that may be used to print biological cells in a 3D structure, some of which may be used or adapted for use with the 3D cell growth medium described herein. The cell growth and interaction apparatus may be operated in some embodiments to implement techniques described below.

Overview

A new class of biomaterial may enable study of tissue cell dynamics: structured 3D cell assemblies in yield stress materials. One of the unique features of the 3D cell assemblies described herein is the substrate in which they are embedded: yield stress materials. The interplay of yield stress, interfacial tension, and cytoskeletal tension may generate new instabilities analogous to those of classical solids and fluids. Yield stress materials may be applied to these studies because (1) their properties allow for unprecedented versatility of cell assembly design, (2) they are homogeneous and transparent, enabling high quality imaging and tractable modeling, and (3) their use with cell assemblies represents the creation of a new class of biomaterial.

Mechanical instabilities in simple structures may be used to classify and measure collective cell forces. The hallmarks of instabilities reveal underlying forces, and to study instabilities is to study the interplay of dominating forces. For example, radial oscillations in fluid jets are the hallmark of the Rayleigh-Plateau instability; measuring these fluctuations probes the interplay of surface tension, viscous stress, and inertia. For cell-assemblies embedded in a support material, the emergent, dominating forces are not known. Simple cell structures may be created in yield stress materials, allowing for investigation of unstable behavior and the hallmarks of classic instabilities. The breadth of structures accessible with the methods described herein may be tested. Stresses may be measured optically by dispersing fluorescent markers in the yield stress material. The threshold of structure stability may be studied by tuning the yield stress of the embedding medium.

The symmetry and topology of complex multicellular structures may have a role in collective cell dynamics. Observations of cells in toroid structures have led us to a guiding discovery: topology can be used for load bearing. The inventors have investigated collective cell dynamics in single loop structures (topological genus=1), and in large arrays of loops (genus >1). The stability of loops depends on the yield stress of the material and cytoskeletal tension, which can be manipulated in many ways. Symmetry of loop arrays may control collective motion; if vorticity develops around each loop, even transiently, loop-loop interactions may arise. The inventors have looked for an anti-ferromagnetic phase in square vortex lattices; spin-glass phases may appear in hexagonal vortex lattices. Stability and correlation are compared between 1D, 2D, and 3D lattices.

In summary, yield stress materials have never been harnessed to create controlled, complex 3D cell structures. The first activities employing this new bio-material may uncover new kinds of mechanical instability arising from the combination of living, self-driven cells with a complex material. This unique combination enables the creation of large, multicellular lattices with which fundamental questions about the roles of symmetry and topology in collective cell behavior can be explored for the first time.

Approach

A conventional paradigm in cellular biomaterials research is to create a solid scaffold and demonstrate its biocompatibility in vitro or in vivo. The properties of extant scaffold systems prohibit versatile experimentation of cell dynamics, limiting investigations of scaffold interactions with living cells. The characteristic shared by these scaffold systems: they are solid. Creating well controlled 3D cell assemblies of arbitrary design in solid scaffolds may not be possible. One question is how one can create a 3D cell manifold inside of a solid scaffold without damaging the scaffold. The use of the yield stress cellular biomaterial is significant because it may (1) create a superior platform for carrying out fundamental investigations of 3D cell dynamics; (2) create a new class of biomaterial never before investigated; and (3) explore fundamental aspects of collective cell dynamics previously prohibited from study, limited by available support materials. These activities are founded on a new concept that breaks with the established paradigm in cellular biomaterials.

Yield stress materials may meet requirements of cellular biomaterials research, such as (1) control of cell aggregate size and shape; (2) measurement of cell generated force; and (3) optical imaging. Yield stress materials (YSMs) are solids when applied stress is below the yield stress, σy. At stresses exceeding σy, YSMs fluidize. When the applied stress falls below σy, a fluidized YSM solidifies again. These properties enable the generation of countless multi-cellular structures by extruding cells or cell/ECM mixtures into YSMs. As the cells are extruded, the nozzle fluidizes the YSM, and when the structure is complete, the extruding nozzle can be removed from the YSM, leaving behind homogeneous support material.

One of the YSMs that may be used is Carbopol, a commercially available material. Carbopol is popular in the study of YSMs because, once yielded, it does not shear thin as strain rate rises, making it perform well for embodiments described herein. MRI velocimetry on Carbopol samples showed that the local strain is the same as the bulk strain across the yielding threshold; this is noteworthy because it demonstrates Carbopol's homogeneity, raising the possibility of developing a 3D force microscopy method.

Instrument Construction

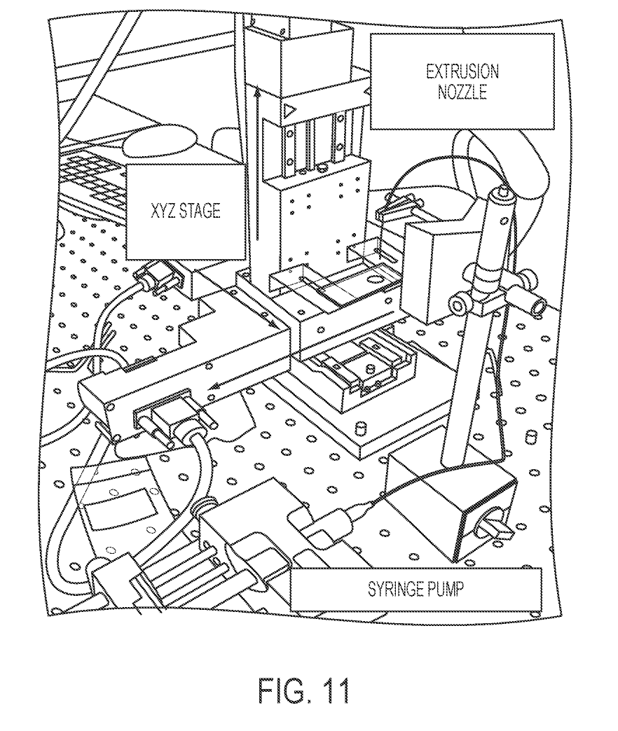

The cell culturing and interaction apparatus of some embodiments may include a 3D extrusion system for depositing cells in a 3D growth medium that is a yield stress material. The extrusion system may comprise an XYZ stage constructed from three linear translation stages (M-403, Physik Instrumente) driven by Mercury DC motor controllers (C-863, Physik Instrumente). The extrusion system may include a computer-controlled syringe pump (Next Advance), held stationary to enable imaging as the stage moves, translating the yield stress support material in 3D (FIG. 11). The extrusion nozzles may include glass pipettes, pulled with a Kopf-750 micropipette puller and shaped with a Narishige micro-forge. The apparatus may include nozzels having various diameters and shapes. Nozzle wettability may be varied automatically and/or manually with hydrophilic 3-aminopropyl-triethoxysilane, or hydrophobic octadecyltriethoxysiloxane.

### Additional Exemplary Implementation of 3D Printing Equipment System

Described below are examples of techniques that may be used to print biological cells in a 3D structure in a manner arrangement as a complex combination of tissues, to replicate a portion of an organism. Some of these techniques may be used or adapted for use with the cell growth and interaction apparatus described herein. The cell growth and interaction apparatus may be operated in some embodiments to implement techniques described below.

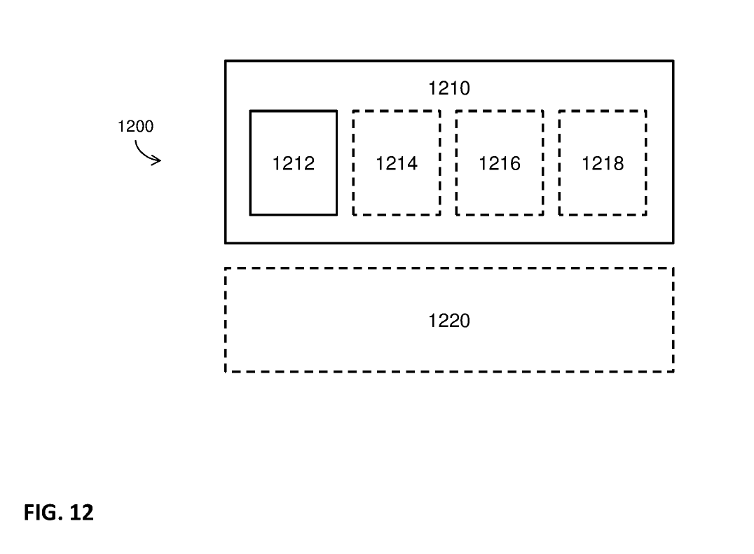

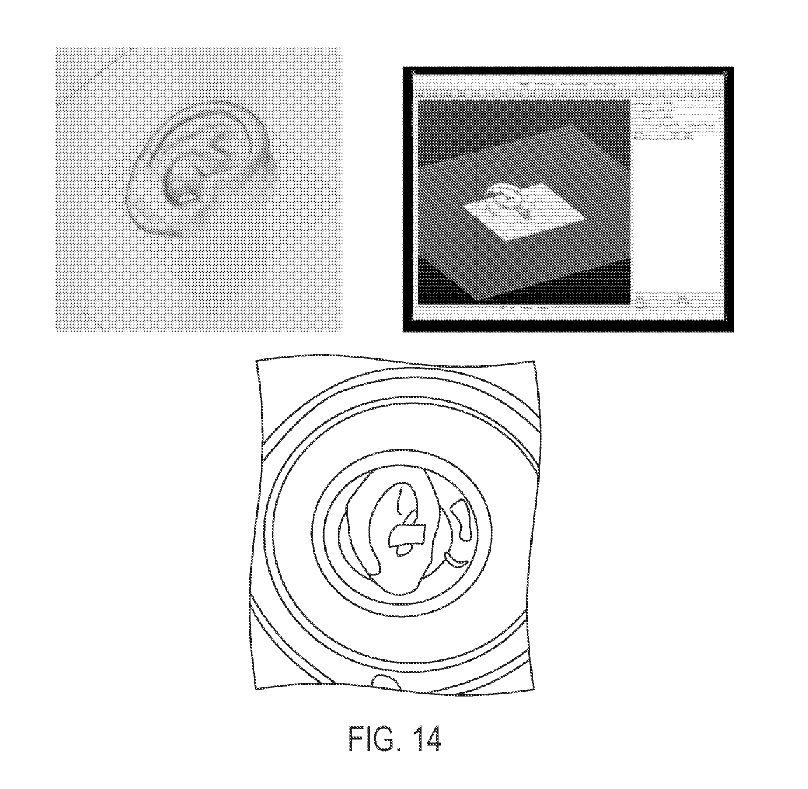

FIG. 12 illustrates an exemplary system 1200 for creating a three-dimensional tissue construct of a desired shape for repair or replacement of a portion of an organism. For example, the tissue construct may be in the shape of and have the characteristics of a human ear (FIG. 14). The tissue construct may then be attached to the organism. The tissue construct may be a tissue repair scaffold, such that the tissue construct may merge with the organism by tissue from the organism growing into the tissue construct.

The system 1200 may include an apparatus 1210. The apparatus 1210 may include an injector 1212. According to some embodiments, the injector 1212 may be configured to inject a biomaterial or multiple biomaterials in a three-dimensional pattern into a first material such that the biomaterial(s) are held in the desired shape of the tissue construct by the first material. The first material may include a yield stress or thixotropic material. According to some embodiments, the injector 1212 may cause a phase change in a region of the first material by applying focused energy to the region using a focused energy source, as described herein. Additionally, the injector 1212 may displace the first material in the region with the biomaterial(s).

According to some embodiments, the apparatus 1210 may also include a removal mechanism 1214, an insertion mechanism 1216, and/or an attachment mechanism 1218. According to some embodiments, the removal mechanism 1214 may be configured to remove the injected biomaterial(s) from within the first material, such as by draining or washing away the first material in whole or in part. The insertion mechanism 1216 may be configured to insert the tissue construct into a wound or tissue void of the organism. The attachment mechanism 1218 may be configured to attach the tissue construct to the organism, such as with adhesive, stitching, suction, precise placement, and/or any other suitable attachment technique. The attachment mechanism 1218 may also be configured to cover the wound or tissue void with flaps of skin or other suitable tissue or material and/or any suitable healing dressing.

According to some embodiments, the apparatus 1210 and/or the system 1200 may also include at least one processor. Additionally, the system 1200 may include a three-dimensional scanner 1220, which may be a laser scanner. The processor(s) may be configured to prepare a model of the tissue construct. The model of the tissue construct may define the shape of the tissue construct as well as the type of material. For example, a tissue construct, designed to merge with a portion of an organism with exposed smooth muscle may be made with biomaterials compatible with smooth muscle. Other portions of the tissue construct may be made of material compatible with other types of tissue in the organism in contact with the tissue construct, such as bone. Similarly, the tissue construct may be designed with openings to align with vasculature in the organism. These parameters of the tissue construct may be represented in the model.

Preparing the model may include scanning a tissue region (e.g., a wound) of the organism that will receive the tissue construct using the three-dimensional scanner 1220. Preparing the model may also include generating the model of the tissue construct so that the tissue construct includes the following: biomaterial serving as a bone replacement adjacent a location of bone identified in the organism, biomaterial serving as a muscle replacement adjacent a location of muscle identified in the organism, and/or biomaterial serving as a vasculature replacement adjacent a location of vasculature identified in the organism. The size and tissue type may be identified through the scanning or in any other suitable way. Alternatively or additionally, preparing the model may include downloading the model from a model repository or any other suitable source.

According to some embodiments, preparing the model of the tissue construct may also include scanning a healthy body part. For example, if one leg of a human subject is wounded or has missing or damaged tissue for any other reason, the processor(s) may use the three-dimensional scanner 1220 to use the human subject's healthy leg, if it is available, as a source of data for preparing the model of the tissue construct. The processor(s) may combine the results of the scanning of the tissue region of the organism with the results of the scanning of the healthy body part (e.g., the healthy leg). The processor(s) may then generate the model of the tissue construct based on the results of this combination. For example, the processor(s) may perform a Boolean operation to determine the differences between the healthy body part and the tissue region of the organism and use these differences as a basis for generating the model of the tissue construct.

According to some embodiments, preparing of the model of the tissue construct may be performed using commercially available software, which may include “off the shelf” scanning, modeling, and/or printing software (FIG. 14). For example, common 3D model file formats may be used to create the tissue construct in common 3D modeling and printing software. The inventors have recognized and appreciated that using commercially available software may reduce the cost and increase the speed of the 3D printing techniques described herein.

According to some embodiments, the biomaterial may be configured to support at least two cell types. For example, the cell types may include bone cell, smooth muscle cell, skeletal muscle cell, vascular cell, and/or any other cell type. Alternatively or additionally, the biomaterial may include at least two material types, which may include material for bone, material for smooth muscle, material for skeletal muscle, material for vasculature, and/or any other suitable material type. The material for bone may include hydroxyapatite based matrices. The material for smooth muscle may include a first hydrogel functionalized with adhesive ligands. The material for skeletal muscle may include a second hydrogel with higher stiffness than the first hydrogel and that is adhesive and configured to stretch periodically. The material for vasculature may include a third hydrogel functionalized with vascular endothelial growth factor.

According to some embodiments, the smallest feature size of the tissue construct may be approximately ten micrometers. For example, the tissue construct may have microscale detail that includes features the size of a microscopic, biological cell.

According to some embodiments, the duration required in creating the tissue construct may be about one hour. Alternatively, the duration required may be a few minutes.

According to some embodiments, high speed manufacturing methods may also be used to increase the speed of creation of a tissue construct and/or inserting the tissue construct into a tissue void.

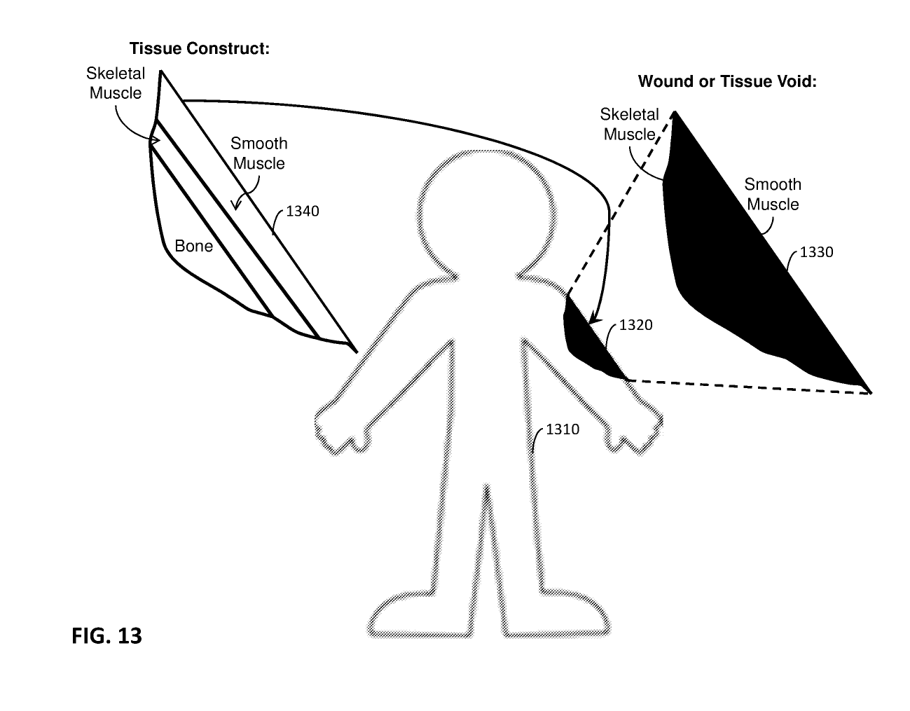

FIG. 13 illustrates an exemplary wound or tissue void of an organism and a tissue construct created to replace or repair the wound or tissue void according to some embodiments. According to some embodiments, the wound or tissue void 1320 may include missing bone tissue, missing skeletal muscle tissue, missing smooth muscle tissue, and/or any other suitable tissue, as illustrated in the enlarged section 1330 of a wound or tissue void 1320 in a human subject's 1310 arm. A tissue construct 1340 may be created with a desired shape for repair or replacement of a portion of an organism, such as this wound or tissue void 1320.

According to some embodiments, the tissue construct 1340 may include multiple biomaterials set in a three-dimensional structure. For example, the biomaterials may include a muscle replacement material and passages for vasculature. Alternatively or additionally, the biomaterials may include two or more of the following: a material that supports growth of bone cell, a material that supports growth of smooth muscle cell, a material that supports growth of skeletal muscle cell, or a material that supports growth of vascular cell.

According to some embodiments, the material that supports growth of bone cell may include hydroxyapatite based matrices. The material that supports growth of smooth muscle cell may include a first hydrogel functionalized with adhesive ligands. The material that supports growth of skeletal muscle cell may include a second hydrogel with higher stiffness than the first hydrogel and that is adhesive and configured to stretch periodically. Alternatively or additionally, the material that supports growth of vascular cells may include a third hydrogel functionalized with vascular endothelial growth factor.

According to some embodiments, the biomaterials may include two or more of the following: a bone replacement material, a smooth muscle replacement material, a skeletal muscle replacement material, or a vasculature replacement material. The bone replacement material may include hydroxyapatite based matrices. The smooth muscle replacement material may include a first hydrogel functionalized with adhesive ligands. The skeletal muscle replacement material may include a second hydrogel with higher stiffness than the first hydrogel and that is adhesive and configured to stretch periodically. Alternatively or additionally, the vasculature replacement material may include a third hydrogel functionalized with vascular endothelial growth factor.

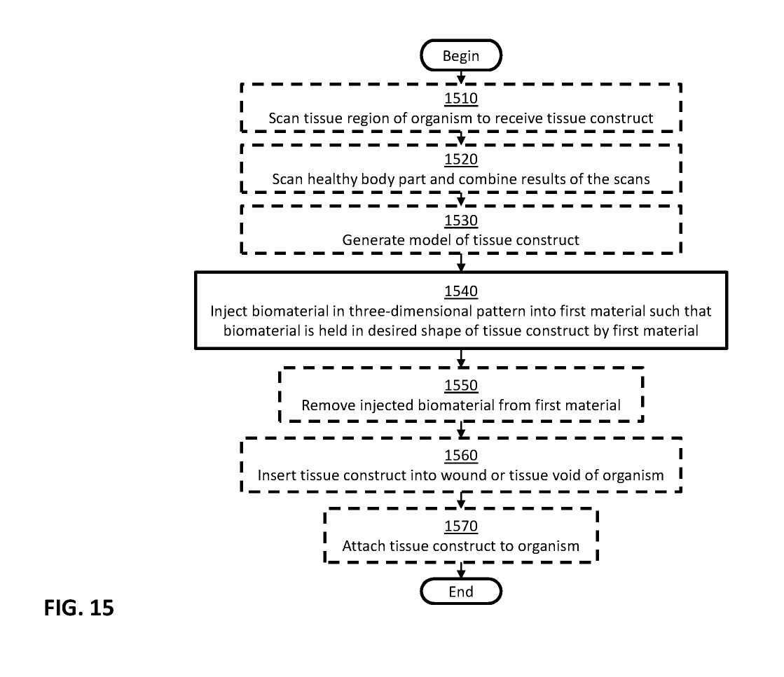

It should be appreciated from the foregoing that some embodiments are directed to a method for three-dimensionally creating a tissue construct, as illustrated in FIG. 15. The method optionally begins at act 1510, at which a tissue region of an organism that is to receive a tissue construct may be scanned. The method then optionally proceeds to act 1520, at which a healthy body part may be scanned and the scan of the tissue region may be combined with the scan of the healthy body part. Optionally, the method then proceeds to act 1530, at which the tissue construct model may be generated.

The method then proceeds to act 1540, at which biomaterial may be injected in a three-dimensional pattern into a first material such that the biomaterial is held in the desired shape of the tissue construct by the first material. Optionally, the method then proceeds to act 1550, at which the injected biomaterial may be removed from the first material. Optionally, the method then proceeds to act 1560, at which the tissue may be inserted into the wound or tissue void of the organism. The method then optionally proceeds to act 1570, at which the tissue construct may be attached to the organism.

The method may then end. However, treatment of the person, or other organism, to which the tissue construct is attached may continue as is known in the art. After the tissue construct is attached to the organism, for example the wound or area of attachment may be periodically irrigated or otherwise treated as is known in the art to promote growth of the tissue from the organism to merge the construct and the organism.

### Computing Environment

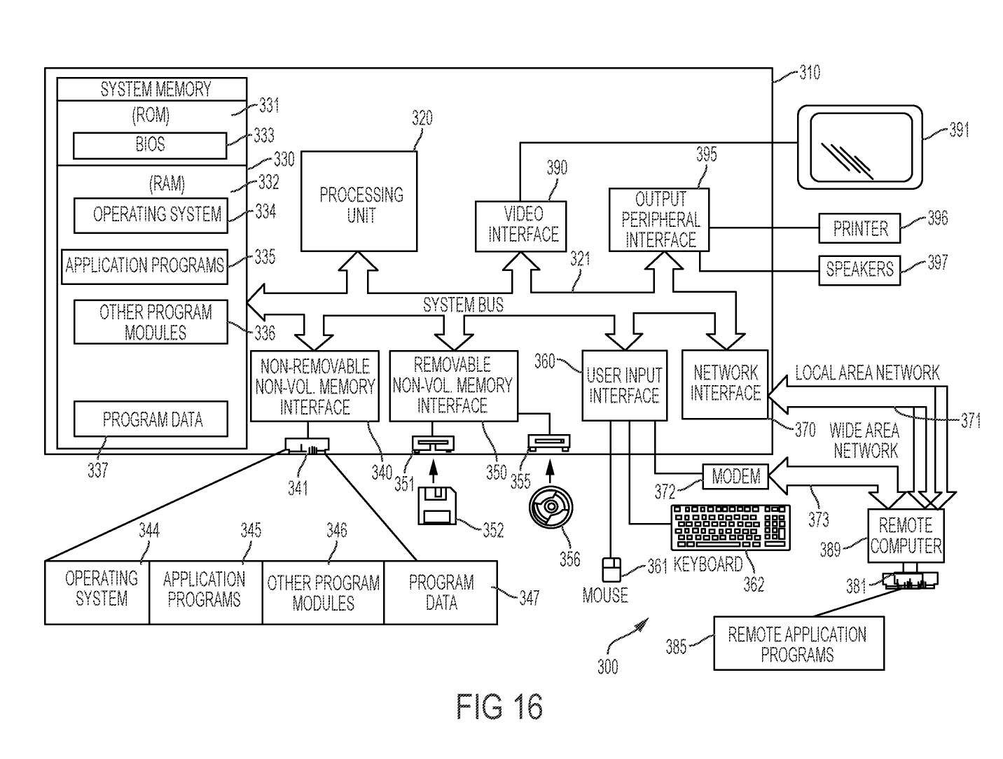

Techniques as described herein may be implemented on any suitable hardware, including a programmed computing system. For example, analysis of a scan and construction of a model may be performed by programming a computing device. Similarly, control of a 3D printing device to print biomaterials in accordance with a model may be controlled by a programmed computing device. FIGS. 1 and 12 illustrate a system that may be implemented with multiple computing devices, which may be distributed and/or centralized. Also, FIGS. 4, 5, 8, and 15 illustrate a process that may include algorithms executing on at least one computing device. FIG. 16 illustrates an example of a suitable computing system environment 300 on which embodiments of these algorithms may be implemented. This computing system may be representative of a computing system that implements the techniques described herein. However, it should be appreciated that the computing system environment 300 is only one example of a suitable computing environment and is not intended to suggest any limitation as to the scope of use or functionality of the invention. Neither should the computing environment 300 be interpreted as having any dependency or requirement relating to any one or combination of components illustrated in the exemplary operating environment 300.

The invention is operational with numerous other general purpose or special purpose computing system environments or configurations. Examples of well-known computing systems, environments, and/or configurations that may be suitable for use with the invention include, but are not limited to, personal computers, server computers, hand-held or laptop devices, multiprocessor systems, microprocessor-based systems, set top boxes, programmable consumer electronics, network PCs, minicomputers, mainframe computers, distributed computing environments or cloud-based computing environments that include any of the above systems or devices, and the like.

The computing environment may execute computer-executable instructions, such as program modules. Generally, program modules include routines, programs, objects, components, data structures, etc. that perform particular tasks or implement particular abstract data types. The invention may also be practiced in distributed computing environments where tasks are performed by remote processing devices that are linked through a communications network. In a distributed computing environment, program modules may be located in both local and remote computer storage media including memory storage devices.

With reference to FIG. 16, an exemplary system for implementing the invention includes a general purpose computing device in the form of a computer 310. Though a programmed general purpose computer is illustrated, it should be understood by one of skill in the art that algorithms may be implemented in any suitable computing device. Accordingly, techniques as described herein may be implemented in any suitable system. These techniques may be implemented in such network devices as originally manufactured or as a retrofit, such as by changing program memory devices holding programming for such network devices or software download. Thus, some or all of the components illustrated in FIG. 36, though illustrated as part of a general purpose computer, may be regarded as representing portions of a node or other component in a network system.

Components of computer 310 may include, but are not limited to, a processing unit 320, a system memory 330, and a system bus 321 that couples various system components including the system memory 330 to the processing unit 320. The system bus 321 may be any of several types of bus structures including a memory bus or memory controller, a peripheral bus, and a local bus using any of a variety of bus architectures. By way of example and not limitation, such architectures include Industry Standard Architecture (ISA) bus, Micro Channel Architecture (MCA) bus, Enhanced ISA (EISA) bus, Video Electronics Standards Association (VESA) local bus, and Peripheral Component Interconnect (PCI) bus also known as Mezzanine bus.

Computer 310 typically includes a variety of computer readable media. Computer readable media can be any available media that can be accessed by computer 310 and includes both volatile and nonvolatile media, removable and non-removable media. By way of example, and not limitation, computer readable media may comprise computer storage media and communication media. Computer storage media includes both volatile and nonvolatile, removable and non-removable media implemented in any method or technology for storage of information such as computer readable instructions, data structures, program modules or other data. Computer storage media includes, but is not limited to, RAM, ROM, EEPROM, flash memory or other memory technology, CD-ROM, digital versatile disks (DVD) or other optical disk storage, magnetic cassettes, magnetic tape, magnetic disk storage or other magnetic storage devices, or any other medium that can be used to store the desired information and that can be accessed by computer 310. Communication media typically embodies computer readable instructions, data structures, program modules, or other data in a modulated data signal such as a carrier wave or other transport mechanism and includes any information delivery media. The term “modulated data signal” means a signal that has one or more of its characteristics set or changed in such a manner as to encode information in the signal. By way of example and not limitation, communication media includes wired media such as a wired network or direct-wired connection, and wireless media such as acoustic, radio frequency (RF), infrared (IR), and other wireless media. Combinations of any of the above should also be included within the scope of computer readable media.

The system memory 330 includes computer storage media in the form of volatile and/or nonvolatile memory such as read only memory (ROM) 331 and random access memory (RAM) 332. A basic input/output system 333 (BIOS), containing the basic routines that help to transfer information between elements within computer 310, such as during start-up, is typically stored in ROM 331. RAM 332 typically contains data and/or program modules that are immediately accessible to and/or presently being operated on by processing unit 320. By way of example and not limitation, FIG. 16 illustrates operating system 334, application programs 335, other program modules 336, and program data 337.

The computer 310 may also include other removable/non-removable, volatile/nonvolatile computer storage media. By way of example only, FIG. 16 illustrates a hard disk drive 341 that reads from or writes to non-removable, nonvolatile magnetic media, a magnetic disk drive 351 that reads from or writes to a removable, nonvolatile magnetic disk 352, and an optical disk drive 355 that reads from or writes to a removable, nonvolatile optical disk 356 such as a CD-ROM or other optical media. Other removable/non-removable, volatile/nonvolatile computer storage media that can be used in the exemplary operating environment include, but are not limited to, magnetic tape cassettes, flash memory cards, digital versatile disks, digital video tape, solid state RAM, solid state ROM, and the like. The hard disk drive 341 is typically connected to the system bus 321 through a non-removable memory interface such as interface 340, and magnetic disk drive 351 and optical disk drive 355 are typically connected to the system bus 321 by a removable memory interface, such as interface 350.

The drives and their associated computer storage media discussed above and illustrated in FIG. 16, provide storage of computer readable instructions, data structures, program modules, and other data for the computer 310. In FIG. 16, for example, hard disk drive 341 is illustrated as storing operating system 344, application programs 345, other program modules 346, and program data 347. Note that these components can either be the same as or different from operating system 334, application programs 335, other program modules 336, and program data 337. Operating system 344, application programs 345, other program modules 346, and program data 347 are given different numbers here to illustrate that, at a minimum, they are different copies. A user may enter commands and information into the computer 310 through input devices such as a keyboard 362 and pointing device 361, commonly referred to as a mouse, trackball, or touch pad. Other input devices (not shown) may include a microphone, joystick, game pad, satellite dish, scanner, or the like. These and other input devices are often connected to the processing unit 320 through a user input interface 360 that is coupled to the system bus, but may be connected by other interface and bus structures, such as a parallel port, game port, or a universal serial bus (USB). A monitor 391 or other type of display device is also connected to the system bus 321 via an interface, such as a video interface 390. In addition to the monitor, computers may also include other peripheral output devices such as speakers 397 and printer 396, which may be connected through an output peripheral interface 395.

The computer 310 may operate in a networked environment using logical connections to one or more remote computers, such as a remote computer 380. The remote computer 380 may be a personal computer, a server, a router, a network PC, a peer device, or some other common network node, and typically includes many or all of the elements described above relative to the computer 310, although only a memory storage device 381 has been illustrated in FIG. 16. The logical connections depicted in FIG. 16 include a local area network (LAN) 371 and a wide area network (WAN) 373, but may also include other networks. Such networking environments are commonplace in offices, enterprise-wide computer networks, intranets, and the Internet.

When used in a LAN networking environment, the computer 310 is connected to the LAN 371 through a network interface or adapter 370. When used in a WAN networking environment, the computer 310 typically includes a modem 372 or other means for establishing communications over the WAN 373, such as the Internet. The modem 372, which may be internal or external, may be connected to the system bus 321 via the user input interface 360, or other appropriate mechanism. In a networked environment, program modules depicted relative to the computer 310, or portions thereof, may be stored in the remote memory storage device. By way of example and not limitation, FIG. 16 illustrates remote application programs 385 as residing on memory device 381. It will be appreciated that the network connections shown are exemplary and other means of establishing a communications link between the computers may be used.

Having thus described several aspects of at least one embodiment of this invention, it is to be appreciated that various alterations, modifications, and improvements will readily occur to those skilled in the art.

For example, techniques are described in which biomaterials are printed into a first material that temporarily changes for a more solid to a more fluid phase upon introduction of energy. Alternatively, materials that become less fluid upon introduction of energy, such as polymers that cure, might also be used.

As another example, biomaterials containing polymers that may be cross-linked or otherwise cured were described as a way to make a tissue construct with structural integrity from material injected in a liquid phase into the first material. Other materials that can be injected in a fluid state, and converted to a material with structural integrity may alternatively or additionally be used. For example, tissue culture medium, containing live cells that may grow and adhere to one another may alternatively or additionally be used.

Such alterations, modifications, and improvements are intended to be part of this disclosure, and are intended to be within the spirit and scope of the invention. Further, though advantages of the present invention are indicated, it should be appreciated that not every embodiment of the invention will include every described advantage. Some embodiments may not implement any features described as advantageous herein and in some instances. Accordingly, the foregoing description and drawings are by way of example only.

The above-described embodiments of the present invention can be implemented in any of numerous ways. For example, the embodiments may be implemented using hardware, software or a combination thereof. When implemented in software, the software code can be executed on any suitable processor or collection of processors, whether provided in a single computer or distributed among multiple computers. Such processors may be implemented as integrated circuits, with one or more processors in an integrated circuit component. Though, a processor may be implemented using circuitry in any suitable format.

Further, it should be appreciated that a computer may be embodied in any of a number of forms, such as a rack-mounted computer, a desktop computer, a laptop computer, or a tablet computer. Additionally, a computer may be embedded in a device not generally regarded as a computer but with suitable processing capabilities, including a Personal Digital Assistant (PDA), a smart phone or any other suitable portable or fixed electronic device.

Also, a computer may have one or more input and output devices. These devices can be used, among other things, to present a user interface. Examples of output devices that can be used to provide a user interface include printers or display screens for visual presentation of output and speakers or other sound generating devices for audible presentation of output. Examples of input devices that can be used for a user interface include keyboards, and pointing devices, such as mice, touch pads, and digitizing tablets. As another example, a computer may receive input information through speech recognition or in other audible format.

Such computers may be interconnected by one or more networks in any suitable form, including as a local area network or a wide area network, such as an enterprise network or the Internet. Such networks may be based on any suitable technology and may operate according to any suitable protocol and may include wireless networks, wired networks or fiber optic networks.

Also, the various methods or processes outlined herein may be coded as software that is executable on one or more processors that employ any one of a variety of operating systems or platforms. Additionally, such software may be written using any of a number of suitable programming languages and/or programming or scripting tools, and also may be compiled as executable machine language code or intermediate code that is executed on a framework or virtual machine.

In this respect, the invention may be embodied as a computer readable storage medium (or multiple computer readable media) (e.g., a computer memory, one or more floppy discs, compact discs (CD), optical discs, digital video disks (DVD), magnetic tapes, flash memories, circuit configurations in Field Programmable Gate Arrays or other semiconductor devices, or other tangible computer storage medium) encoded with one or more programs that, when executed on one or more computers or other processors, perform methods that implement the various embodiments of the invention discussed above. As is apparent from the foregoing examples, a computer readable storage medium may retain information for a sufficient time to provide computer-executable instructions in a non-transitory form. Such a computer readable storage medium or media can be transportable, such that the program or programs stored thereon can be loaded onto one or more different computers or other processors to implement various aspects of the present invention as discussed above. As used herein, the term “computer-readable storage medium” encompasses only a computer-readable medium that can be considered to be a manufacture (i.e., article of manufacture) or a machine. Alternatively or additionally, the invention may be embodied as a computer readable medium other than a computer-readable storage medium, such as a propagating signal.

The terms “program” or “software” are used herein in a generic sense to refer to any type of computer code or set of computer-executable instructions that can be employed to program a computer or other processor to implement various aspects of the present invention as discussed above. Additionally, it should be appreciated that according to one aspect of this embodiment, one or more computer programs that when executed perform methods of the present invention need not reside on a single computer or processor, but may be distributed in a modular fashion amongst a number of different computers or processors to implement various aspects of the present invention.

Computer-executable instructions may be in many forms, such as program modules, executed by one or more computers or other devices. Generally, program modules include routines, programs, objects, components, data structures, etc. that perform particular tasks or implement particular abstract data types. Typically the functionality of the program modules may be combined or distributed as desired in various embodiments.

Also, data structures may be stored in computer-readable media in any suitable form. For simplicity of illustration, data structures may be shown to have fields that are related through location in the data structure. Such relationships may likewise be achieved by assigning storage for the fields with locations in a computer-readable medium that conveys relationship between the fields. However, any suitable mechanism may be used to establish a relationship between information in fields of a data structure, including through the use of pointers, tags or other mechanisms that establish relationship between data elements.

Various aspects of the present invention may be used alone, in combination, or in a variety of arrangements not specifically discussed in the embodiments described in the foregoing and is therefore not limited in its application to the details and arrangement of components set forth in the foregoing description or illustrated in the drawings. For example, aspects described in one embodiment may be combined in any manner with aspects described in other embodiments.

Also, the invention may be embodied as a method, of which an example has been provided. The acts performed as part of the method may be ordered in any suitable way. Accordingly, embodiments may be constructed in which acts are performed in an order different than illustrated, which may include performing some acts simultaneously, even though shown as sequential acts in illustrative embodiments.

Use of ordinal terms such as “first,” “second,” “third,” etc., in the claims to modify a claim element does not by itself connote any priority, precedence, or order of one claim element over another or the temporal order in which acts of a method are performed, but are used merely as labels to distinguish one claim element having a certain name from another element having a same name (but for use of the ordinal term) to distinguish the claim elements.

Also, the phraseology and terminology used herein is for the purpose of description and should not be regarded as limiting. The use of “including,” “comprising,” or “having,” “containing,” “involving,” and variations thereof herein, is meant to encompass the items listed thereafter and equivalents thereof as well as additional items.

In the attached claims, various elements are recited in different claims. However, the claimed elements, even if recited in separate claims, may be used together in any suitable combination.

1. An apparatus for culturing and interacting with biological cells and/or tissues, the apparatus comprising:
a chamber comprising a container holding a three-dimensional (3D) cell growth medium, the 3D cell growth medium being a thixotropic material;
equipment to dispense biological cells and/or tissues at particular positions within the 3D cell growth medium in the container; and
equipment to interact with the biological cells and/or tissues within the 3D cell growth medium in the container.

2. The apparatus of claim 1, further comprising:
equipment to dispense the 3D cell growth medium into the container.

3. The apparatus of claim 1, wherein the 3D cell growth medium comprises:
a plurality of hydrogel particles; and
a liquid cell culture medium,
wherein the hydrogel particles are swelled with the liquid cell culture medium to form a granular gel.

4. The apparatus of claim 3, wherein the concentration of hydrogel particles is between 0.05% to 1.0% by weight.

5. The apparatus of claim 3, wherein the hydrogel particles have a size in the range of 0.1 μm to 100 μm when swollen with the liquid cell culture medium.

6. The apparatus of claim 5, wherein the hydrogel particles have a size in the range of 1 μm to 10 μm when swollen with the liquid cell culture medium.

7. The apparatus of claim 1, wherein the equipment to interact with the biological cells and/or tissues within the 3D cell growth medium in the container comprises equipment to add, remove, and/or exchange fluid materials in the chamber.

8. The apparatus of claim 7, further comprising:
a bioreactor, wherein the bioreactor comprises the chamber and the equipment to interact.

9. The apparatus of claim 7, further comprising:
a controller to operate the equipment to interact with the biological cells and/or tissues within the 3D cell growth medium, wherein the controller is configured to move at least some of the equipment to interact to penetrate the 3D cell growth medium following dispensing of the biological cells and/or tissues by the equipment to dispense.

10. The apparatus of claim 1, wherein the equipment to interact with the biological cells and/or tissues within the 3D cell growth medium in the container comprises equipment to dispense a material in the chamber and/or in the 3D cell growth medium while the biological cells and/or tissues are disposed in the 3D cell growth medium.

11. The apparatus of claim 10, wherein the equipment to dispense a material in the chamber and/or in the 3D cell growth medium comprises equipment to dispense nutrients for the biological cells and/or tissues.

12. The apparatus of claim 10, wherein the equipment to dispense a material in the chamber and/or in the 3D cell growth medium comprises equipment to dispense a pharmaceutical or a combination of pharmaceuticals in the chamber and/or in the 3D cell growth medium.

13. The apparatus of claim 1, wherein the equipment to interact with the biological cells and/or tissues within the 3D cell growth medium in the container comprises equipment to remove a material from the chamber and/or from the 3D cell growth medium while the biological cells and/or tissues are disposed in the 3D cell growth medium.

14. The apparatus of claim 13, wherein the equipment to remove a material from the chamber and/or from the 3D cell growth medium comprises equipment to remove waste from the chamber and/or the 3D cell growth medium.

15. The apparatus of claim 13, wherein the equipment to remove a material from the chamber and/or from the 3D cell growth medium comprises equipment to remove from the chamber and/or the 3D cell growth medium a byproduct created by the biological cells and/or tissues.

16. A method of operating a bioreactor, the method comprising:
culturing cells and/or tissues in a 3D cell growth medium, the 3D cell growth medium being a thixotropic material; and
while the cells and/or tissues are disposed in the 3D cell growth medium, removing byproduct of cellular activity from the 3D cell growth medium.

17. The method of claim 16, further comprising:
while the cells and/or tissues are disposed in the 3D cell growth medium, replenishing the 3D cell growth medium.

18. (canceled)

19. (cancelled)

20. (canceled)

21. (cancelled)

22. The method of claim 16, further comprising:
while the cells and/or tissues are disposed in the 3D cell growth medium, supplying a compound to the cells and/or tissues.

23. The method of claim 22, wherein supplying the compound to the cells and/or tissues comprises dispensing the compound in the 3D cell growth medium in an area adjacent to the cells and/or tissues.

24. The method of claim 22, wherein supplying the compound to the cells and/or tissues comprises dispensing the compound in an area of the 3D cell growth medium to enable the compound to diffuse across the 3D cell growth medium from the area to the cells and/or tissues.

25. The method of claim 22, wherein supplying the compound to the cells and/or tissues comprises supplying a first compound to a first portion of the cells and/or tissues and supplying a second compound to a second portion of the cells and/or tissues.

26. The method of claim 25, wherein:
the first compound is a solution comprising a first material in a first concentration; and
the second compound is a solution comprising the first material in a second concentration.

27. The method of claim 22, further comprising:
assaying the cells and/or tissues while the cells and/or tissues are disposed in the 3D cell growth medium, wherein the assaying comprises the supplying the compound.

28. The method of claim 22, wherein supplying the compound comprises supplying one or more materials from a group consisting of: a nutrient, a stain, a fixative, and a pharmaceutical.

29. A method of operating a bioreactor to expose cells to a material, the method comprising:
suspending cells at locations within a 3D cell growth medium contained in a container of the bioreactor, the 3D cell growth medium being a thixotropic material;
operating the bioreactor to culture the cells suspended in the 3D cell growth material;
operating the bioreactor to dispense the material into the 3D cell growth medium; and
following dispensing of the material, evaluating the cells for an impact of the dispensed material on the cells.

30. The method of claim 29, wherein evaluating the cells comprises evaluating the cells while the cells are suspended in the 3D cell growth medium.

31. The method of claim 30, wherein evaluating the cells comprises inserting into the 3D cell growth medium equipment to evaluate the cells.

32. The method of claim 29, wherein:
operating the bioreactor to dispense the material comprises operating the bioreactor to dispense the material so as to create a gradient of concentration of the material in the 3D cell growth medium and expose different cells to different concentrations of the material; and evaluating the cells for the impact of the dispensed material comprises evaluating the cells based on a position of the cells within the gradient.

33. The method of claim 29, wherein:
the 3D cell growth medium comprises a hydrogel and a cell growth material; and
operating the bioreactor to culture the cells comprises adding cell growth material to the 3D cell growth medium during the culturing.

34. The method of claim 33, wherein operating the bioreactor to culture the cells comprises removing from the 3D cell growth medium waste created by the cells.

35. The method of claim 34, wherein removing the waste from the 3D cell growth medium comprises operating a pump and/or a centrifuge to impose a force on the 3D cell growth medium.

36. The method of claim 29, wherein suspending cells at locations within a 3D cell growth medium comprises creating a 3D cell culture by dispensing cells at the locations within the 3D cell growth medium.
